(19) United States
(12) Patent Application Publication
(10) Pub. No.: US 2021/0316088 A1
(43) Pub. Date: Oct. 14, 2021

(54) DEVICES AND METHODS FOR DELIVERING A SUBSTANCE TO A BODY CAVITY

(71) Applicant: SipNose Ltd., Yokneam (IL)

(72) Inventors: Daniel SHAHAF, M.P. Emek Ha-Yarden (IL); Iris SHICHOR, Zichron Ya'akov (IL); Liron Shavit HADAR, Haifa (IL); Lia KAUFMAN, Zichron Ya'akov (IL)

(73) Assignee: SipNose Ltd., Yokneam (IL)

(21) Appl. No.: 17/357,810

(22) Filed: Jun. 24, 2021

Related U.S. Application Data
(63) Continuation-in-part of application No. 16/809,994, filed on Mar. 5, 2020.
Application No. 16/809,994 is a continuation-in-part of application No. 15/982,996, filed on May 17, 2018.
Application No. 15/982,996 is a continuation-in-part of application No. 14/733,143, filed on Jun. 8, 2015.
Application No. 14/733,143 is a continuation-in-part of application No. PCT/IB2021/051834, filed on Mar. 4, 2021.
(60) Provisional application No. 63/113,833, filed on Nov. 14, 2020.
(60) Provisional application No. 62/526,386, filed on Jun. 29, 2017.
(60) Provisional application No. 62/077,246, filed on Nov. 9, 2014.
(60) Provisional application No. 62/117,986, filed on Feb. 19, 2015.

(30) Foreign Application Priority Data
Dec. 16, 2013 (DE) 202013105715.0

(51) Int. Cl. A61M 15/00; A61M 11/00

(52) U.S. Cl. CPC A61M 15/0003; A61M 2202/064; A61M 11/001; A61M 2210/0612; A61M 2210/0618; A61M 2210/065; A61M 2210/0662; A61M 2210/1089; A61M 2202/0275; A61M 2202/0208; A61M 2202/0225; A61M 2202/025; A61M 2202/0291; A61M 2202/04; A61M 15/0036

(57) ABSTRACT

Methods and devices for delivering one or more substances within at least one body cavity are provided. The device includes at least one nosepiece including at least one capsule having a volume V_{sub} of the substances; at least one base in communication with the nosepiece, the base including at least one chamber configured to confine pressurized fluid at volume V_{PF} and pressure P_{PF}; and at least one hollow puncturing member. The hollow puncturing member includes at least one end in fluid communication with the nosepiece and a second end in fluid communication with the base. The first and second ends are fluidly interconnected by a hollow tube. The fluid inlet port of the capsule is configured in terms of size and shape to interface in a sealable manner with the one end of the at least one puncturing member.

### CROSS REFERENCE TO RELATED APPLICATIONS

This application is a Continuation-in-Part of U.S. application Ser. No. 16/809,994, filed on Mar. 5, 2020, which is a Continuation-in-Part of U.S. application Ser. No. 15/982,996 filed on May 17, 2018, which is a Continuation-in-Part of U.S. application Ser. No. 14/733,143 filed on Jun. 8, 2015, which claims the benefit of and priority to U.S. Provisional Application No. 62/117,986 filed on Feb. 19, 2015, and U.S. Provisional Application No. 62/077,246 filed on Nov. 9, 2014. U.S. application Ser. No. 15/982,996 claims further priority to U.S. Provisional Application No. 62/526,386 filed on Jun. 29, 2017.

U.S. application Ser. No. 16/809,994 is also a Continuation-in-Part of U.S. application Ser. No. 16/810,096 filed Mar. 5, 2020, which is a Continuation-in-Part of U.S. application Ser. No. 15/982,630 filed on May 17, 2018, which is a Continuation-in-Part of U.S. application Ser. No. 14/733,143 filed on Jun. 8, 2015, which claims the benefit of and priority to U.S. Provisional Application No. 62/117,986 filed on Feb. 19, 2015, and U.S. Provisional Application No. 62/077,246 filed on Nov. 9, 2014. U.S. application Ser. No. 15/982,630 further claims priority to U.S. Provisional Application No. 62/507,816 filed on May 18, 2017.

Further, U.S. application Ser. No. 16/809,994 is a Continuation-in-Part of U.S. application Ser. No. 14/433,048 filed on Apr. 2, 2015, which is a National Phase Entry of PCT/IL2014/050752 filed on Aug. 21, 2014, which claims the benefit of and priority to U.S. Provisional Application No. 61/868,614 filed on Aug. 22, 2013, and U.S. Provisional Application No. 61/868,627 filed on Aug. 22, 2013, and further priority to German Application No. 2020131057150 filed on Dec. 16, 2013.

Additionally, this application claims priority to and the benefit of U.S. Provisional Application No. 63/113,833 filed on Nov. 14, 2020.

This application is a Continuation-in-Part of International Application No. PCT/IB2021/051834 filed on Mar. 4, 2021.

The contents of the above applications are all incorporated by reference as if fully set forth herein in their entirety.

### FIELD OF THE INVENTION

The present invention generally pertains to a system and methods for delivering aerosolized substance to a natural orifice of the body.

### BACKGROUND OF THE INVENTION

Blow-Fill-Seal (BFS) technology is a manufacturing technique used to produce small (0.1 ml) and large (and up to 500 ml) liquid-filled containers. The basic concept of blow-fill-seal and form-fill-seal (referred to interchangeably hereinafter as BFS) is that a container is formed, filled and sealed in a continuous process without human intervention in a sterile or aseptic enclosed area. Thus, this technology can be used to sterile or aseptically packaging and manufacturing of pharmaceutical liquid dosage forms. Furthermore, this can be applied to any drug container (produced by any production methods) with a laminate at the bottom thereof, which can be pierced (or break) so as to deliver the medicament.

There are several ways of manufacturing. According to one method, the processes begun as pharmaceutical grade plastic resin is vertically heat-extruded through a circular throat to form a hanging tube (parison). This extruded tube is then enclosed within a two-part mold and the tube is cut above the mold. The mold is zone, or a sterile filling space, where filling needles (mandrels) are lowered and used to inflate the plastic to form a container within the mold. Following formation of the container, the mandrel is used to fill the container with the liquid. Following filling, the mandrels are retracted and a secondary top mold seals the container. All actions take place within the sterile enclosed area, a sterile shrouded chamber within the machine. The product can then be discharged to a non-sterile area for labeling, packaging and distribution.

BFS technology reduces personnel intervention, making it a more robust method for aseptic preparation of sterile pharmaceuticals. BFS is used for the filling of vials for parenteral preparations and infusion, eye drops, and inhalation products. Generally, the containers are made of polyethylene and polypropylene or any plastic resin.

It is therefore a long felt need to provide a system which provides an efficient delivery of a substance to a target site from such BFS containers, provides sufficient material to the target site, and ensures reproducibility.

### SUMMARY OF THE INVENTION

It is one object of the present invention to provide a device for delivering either one or more substances within at least one body cavity, comprising:
(i) at least one nosepiece comprising at least one capsule comprising V_{sub} [ml or mg] of said substances; said capsule having at least one fluid inlet port of diameter Dᵢₙ [mm] and at least one fluid discharging outlet of diameter Dₒᵤₜ [mm], configured for placement in proximity to said body cavity;
(ii) at least one base in communication with said at least one nosepiece, said at least one base comprises at least one chamber configured to confine compress and pressurized fluid at volume V_{PF} [ml] and pressure P_{PF} [barg];
(iii) at least one hollow puncturing member; said at least one hollow puncturing member is characterized by at least one end in fluid communication with said at least one capsule in said nosepiece and a second end in fluid communication with said at least one base; said first and second ends are fluidly interconnected by a hollow tube; said fluid inlet port of said capsule is configured by means of size and shape to interface in a sealable manner with said one end of said at least one puncturing member;
wherein actuation of said base configured to enable piercing of said chamber, by means of said puncturing member to enables the pressurized fluid to exit said chamber and entrain through said hollow tube of said hollow puncturing member to said capsule, entrain said substance and deliver the same to said body cavity.

It is another object of the present invention to provide the device as defined above, wherein said chamber is made of material being a high barrier film.

It is another object of the present invention to provide the device as defined above, wherein said high barrier film is a high barrier Aluminum film.

It is another object of the present invention to provide the device as defined above, wherein actuation of said base is pressing on the same.

It is another object of the present invention to provide the device as defined above, wherein said nosepiece comprising at least one nosepiece cover configured by means of size and shape to cover, in a sealable manner, at least partially said nosepiece

It is another object of the present invention to provide the device as defined above, wherein said nosepiece cover and said nosepiece are coupled to each other.

It is another object of the present invention to provide the device as defined above, wherein said coupling between nosepiece cover and said nosepiece said is reversible.

It is another object of the present invention to provide the device as defined above, wherein said nosepiece comprises at least one port throughout which said at least one substance exits said device, such that said nosepiece cover seals said at least one port and removal thereof removes said seal.

It is another object of the present invention to provide the device as defined above, wherein said capsule is selected from a group consisting of pierceable container, a blow-fill-seal and a form-fill-seal and any combination thereof.

It is another object of the present invention to provide the device as defined above, wherein said at least one chamber is a container adapted to hold said pressured fluid at said P_{PF} for prolong periods of time.

It is another object of the present invention to provide the device as defined above, wherein capsule is made of at least one material selected from a group consisting of high- or low-density polyethylene, high- or low-density polypropylene, any plastic resin, glass and any combination thereof.

It is another object of the present invention to provide the device as defined above, wherein said one end of said at least one hollow puncturing member and said capsule are formed as a single unit, such that the lower surface of said capsule is integrated with said one end of said at least one hollow puncturing member.

It is another object of the present invention to provide the device as defined above, wherein said volume V_{PF} [ml] of said pressurized fluid at pressure P_{PF} [barg] is released from said chamber within a short period of time, <500 milliseconds (dT), via said fluid inlet port, entrains said substances, erupts via said fluid discharging outlet port into said body cavity, such that the release time of said V_{sub} [ml or mg] of said substances and said V_{PF} [ml] of said pressurized fluid, dTᵣₑₗₑₐₛₑ is less than 500 milliseconds.

It is another object of the present invention to provide the device as defined above, wherein said device is configured to deliver said V_{sub} substance and V_{PF} pressurized fluid through said fluid discharging outlet of diameter D [mm] wherein at least one of the following is held true:
a. V_{PF} is in a range of 1 to 50 ml;
b. V_{sub} is in a range of about 0.01 to about 7 ml;
c. Dᵢₙ and/or Dₒᵤₜ is in a range of 0.2 to 6 mm;
d. P_{PF} is in a range of about 0 to about 10 barg;
e. said pressure rate,

$$\frac{dP}{dT} \to ∞ ;$$

f. said pressure velocity is greater than 0.001 barg/ms;
g. said pressure velocity is greater than 0.01 barg/ms;
h. said volume rate dV_{sub}/dT or dV_{sub}/dTᵣₑₗₑₐₛₑ is greater than 0.0001 ml/ms;
i. said volume rate dV_{sub}/dT or dV_{sub}/dTᵣₑₗₑₐₛₑ is greater than 0.001 ml/ms;
j. said volume rate dV_{PF}/dT or dV_{PF}/dTᵣₑₗₑₐₛₑ is greater than 0.001 ml/ms;
k. said volume rate dV_{PF}/dT or dV_{PF}/dTᵣₑₗₑₐₛₑ is greater than 0.01 ml/ms;
l. any combination thereof.

It is another object of the present invention to provide the device as defined above, wherein at least one of the following is true:
a. said body cavity is selected from a group consisting of nasal cavity, the mouth, the throat, an ear, the eye, the vagina, the rectum, the urethra, and any combination thereof.
b. said pressurized gas is selected from a group consisting of air, nitrogen, oxygen, carbon dioxide, helium, neon, xenon, nitric oxide and any combination thereof;
c. during dispensing of said at least one substance, a mixture of said predetermined volume V_{gas} [ml] of said pressurized gas with said predetermined volume V_{sub} [ml or mg] of said substance entrained within it forms a plume of aerosol; said aerosol having a predetermined distribution, said distribution being either homogeneous or heterogeneous, said heterogeneous distribution is selected from a group consisting of: an arbitrary distribution, a distribution in which the density of said at least one substance within said mixture follows a predetermined pattern, and any combination thereof; characteristics of said aerosol selected from a group consisting of: particle size, particle shape, particle distribution, and any combination thereof, are determinable from characteristics of said device selected from a group consisting of: said predetermined volume of said pressurized gas, said predetermined volume of said substance, said predetermined pressure of said pressurized gas, said predetermined orifice size, and any combination thereof;
d. at least one said substance is selected from a group consisting of a gas, a liquid, a powder, an aerosol, a slurry, a gel, a suspension and any combination thereof;
e. at least one said substance is stored under either an inert atmosphere or under vacuum to prevent reactions during storage;
f. a dose-response curve is substantially linear for brain concentration of said substance when administered nasally via said device; and
g. a dose-response curve for brain concentration having a fit selected from a group consisting of logarithmic, parabolic, exponential, sigmoid, power-low, and any combination thereof; of said substance when administered nasally via said device.

It is another object of the present invention to provide the device as defined above, wherein said nosepiece cover configured to provide an air-tight closure for said port, said port cover slidable along said device, rotatable around said device, rotatable around a hinge on the exterior of said device and any combination thereof.

It is another object of the present invention to provide the device as defined above, further comprising a safety latch, adapted to prevent accidental operation of said device.

It is another object of the present invention to provide the device as defined above, wherein said substance is selected from a group consisting of proteins; stem-cells; cells, organs, portions, extracts, and isolations thereof; macro-molecules; RNA or other genes and proteins-encoding materials; neurotransmitters; receptor antagonists; hormones; Ketamine; Baqsimi product commercially available by Lilly (US); Glucagon; substrates to treat one of eth followings: anaphylaxis, Parkinson, seizures and opioid overdose; epinephrine; atropine; metoclopramide; commercially available Naloxone or Narcan products; Esketamine (Spravato); Radicava [edaravone]; Ingrezza [valbenazine]; Austedo [deutetrabenazine]; Ocrevus [ocrelizumab]; Xadago [safinamide]; Spinraza [nusinersen]; Zinbryta [daclizumab]; Nuplazid [pimavanserin]; Aristada [aripiprazole lauroxil]; Vraylar [cariprazine]; Rexulti [brexpiprazole]; Aptiom [eslicarbazepine acetate]; Vizamyl [flutemetamol F18 injection]; Brintellix [vortioxetine]; Tecfidera [dimethyl fumarate]; Dotarem [gadoterate meglumine]; Antibody mediated brain targeting drug delivery including aducanumab, gantenerumab, bapineuzumab, solanezumab, ofatumumab CD20, BIIB033, LCN2, HMGB1; insulin; oxytocin; orexin-A; leptin; benzodiazepine, midazolam; naloxone; perillyl alcohol; camptothecin; phytochemicals including curcumin and chrysin; nucleotides; olanzapine; risperidone; Venlafaxin; GDF-5; zonisamide; ropinirole; plant-originated and synthetically-produced terpenes and cannabinoids, including THC and CBD; valproric acid; rivastigmine; estradiol; topiramate or an equivalent preparation comprising CAS No. 97240-79-4; MFSD2 or MFSD2A or sodium-dependent lysophosphatidylcholine symporter, midazolam; naloxone; perillyl alcohol; camptothecin; phytochemicals including curcumin and chrysin; nucleotides; olanzapine; risperidone; Venlafaxin; GDF-5; zonisamide; ropinirole; plant-originated and synthetically-produced terpenes and cannabinoids, including THC and CBD; valproric acid; rivastigmine; estradiol; topiramate or an equivalent preparation comprising CAS No. 97240-79-4; MFSD2 or MFSD2A or sodium-dependent lysophosphatidylcholine symporter; and any esters, salts, derivatives, mixtures, combinations thereof, with or without a carrier, liposomes, lyophilic or water-miscible solvents, surfactants, cells, cells fractions, at a therapeutically effective concentration.

It is another object of the present invention to provide the device as defined above, wherein said capsule is a hollow tube characterized by at least two ends interconnect to each other, at least one of which is positioned proximal to said chamber.

It is another object of the present invention to provide the device as defined above, wherein said capsule comprises at least one spherical element positioned by at least one of said ends, adapted to seal said capsule and prevent leakage of said at least one substrate therefrom.

It is another object of the present invention to provide the device as defined above, wherein said capsule comprises two spherical elements, each of which is disposed at each of said ends, such that said at least one substrate is positioned therebetween.

It is another object of the present invention to provide the device as defined above, wherein said two spherical elements, once said pressurized fluid exits said chamber, are adapted to mix said at least one substrate and said at least one substrate.

It is another object of the present invention to provide the device as defined above, wherein said capsule comprises at least one membrane positioned by at least one of said ends, adapted to seal said capsule and prevent leakage of said at least one substrate therefrom.

It is another object of the present invention to provide the device as defined above, wherein said capsule comprises two membranes, each of which is disposed at each of said ends, such that said at least one substrate is positioned therebetween.

It is another object of the present invention to provide the device as defined above, wherein said capsule comprises at least one duckbill valve positioned by at least one of said ends, adapted to seal said capsule and prevent leakage of said at least one substrate therefrom.

It is another object of the present invention to provide the device as defined above, wherein said capsule comprises two duckbill valves, each of which is disposed at each of said ends, such that said at least one substrate is positioned therebetween.

It is another object of the present invention to provide the device as defined above, wherein said capsule comprises at least one spherical element, membrane, uni-directional valve, duckbill valve and any combination thereof.

It is another object of the present invention to provide a method for delivering either one or more substances within at least one body cavity, characterized by steps of
a. providing:
   i. at least one nosepiece comprising at least one capsule comprising V_{sub} [ml or mg] of said substances; said capsule having at least one fluid inlet port of diameter Dᵢₙ [mm] and at least one fluid discharging outlet of diameter Dₒᵤₜ [mm], configured for placement in proximity to said body cavity;
   ii. at least one base in communication with said at least one nosepiece, said at least one base comprises at least one chamber configured to confine compressed and pressurized fluid at volume V_{PF} [ml] and pressure P_{PF} [barg];
   iii. at least one hollow puncturing member; said at least one hollow puncturing member is characterized by at least one end in fluid communication with said at least one nosepiece and a second end in fluid communication with said at least one base; said first and second ends are fluidly interconnected by a hollow tube; said fluid inlet port of said capsule is configured by means of size and shape to interface in a sealable manner with said one end of said at least one puncturing member;
b. actuating said base thereby piercing of said chamber, by means of said at least one puncturing member.

It is another object of the present invention to provide the method as defined above, wherein said chamber is made of material being high barrier film.

It is another object of the present invention to provide the method as defined above, wherein said high barrier film is a high barrier Aluminum film.

It is another object of the present invention to provide the method as defined above, wherein step (b) of actuating said base additionally comprising pressing said base.

It is another object of the present invention to provide the method as defined above, wherein step (b) of actuating said base enables the pressurized fluid to exit said chamber and entrain through said hollow tube of said hollow puncturing member to said capsule, entrain said substance and deliver the same to said body cavity.

It is another object of the present invention to provide the method as defined above, wherein step (b) of actuating said base results in releasing said volume V_{PF} [ml] of said pressurized fluid at pressure P_{PF} [barg] within a short period of time, <500 milliseconds (dT); out of said chamber, via said fluid inlet thereby entraining said substances and erupting via said fluid discharging outlet into said body cavity, such that the release time of said V_{sub} [ml or mg] of said substances and said V_{PF} [ml] of said pressurized fluid, dTᵣₑₗₑₐₛₑ is less than 500 milliseconds.

It is another object of the present invention to provide the method as defined above, wherein said nosepiece comprising a nosepiece cover and said nosepiece are coupled to each other.

It is another object of the present invention to provide the method as defined above, wherein said coupling between nosepiece cover and said nosepiece said is reversible.

It is another object of the present invention to provide the method as defined above, wherein removal of said nosepiece cover results in piercing said capsule to provide said fluid discharging outlet.

It is another object of the present invention to provide the method as defined above, wherein removal of said nosepiece cover is obtained by at least one action selected from a group consisting of sliding said nosepiece cover along said device, rotating said nosepiece cover around said device, rotating said nosepiece cover around a hinge on the exterior of said device and any combination thereof.

It is another object of the present invention to provide the method as defined above, wherein said nosepiece cover comprises at least one nosepiece puncturing member adapted to pierce said capsule to enable said fluid discharging outlet.

It is another object of the present invention to provide the method as defined above, wherein said nosepiece comprises at least one port throughout which said at least one substance exits said device, such that said nosepiece cover seals said at least one port and removal thereof removes said seal.

It is another object of the present invention to provide the method as defined above, wherein said capsule is selected from a group consisting of pierceable container, a blow-fill-seal and a form-fill-seal and any combination thereof.

It is another object of the present invention to provide the method as defined above, wherein said at least one chamber is a container adapted to hold said pressured fluid at said P_{PF} for prolong periods of time.

It is another object of the present invention to provide the method as defined above, wherein capsule is made of at least one material selected from a group consisting of high- or low-density polyethylene, high- or low-density polypropylene, any plastic resin, glass and any combination thereof.

It is another object of the present invention to provide the method as defined above, wherein said device is configured to deliver said V_{sub} substance and V_{PF} pressurized fluid through said fluid discharging outlet of diameter D [mm] wherein at least one of the following is held true:
a. V_{PF} is in a range of 1 to 50 ml;
b. V_{sub} is in a range of about 0.01 to about 7 ml;
c. Dᵢₙ and/or Dₒᵤₜ is in a range of 0.2 to 6 mm;
d. P_{PF} is in a range of about 0 to about 10 barg;
e. said pressure rate,

$$\frac{dP}{dT} \to ∞ ;$$

f. said pressure velocity is greater than 0.001 barg/ms;
g. said pressure velocity is greater than 0.01 barg/ms;
h. said volume rate dV_{sub}/dT or dV_{sub}/dTᵣₑₗₑₐₛₑ is greater than 0.0001 ml/ms;
i. said volume rate dV_{sub}/dT or dV_{sub}/dTᵣₑₗₑₐₛₑ is greater than 0.001 ml/ms;
j. said volume rate dV_{PF}/dT or dV_{PF}/dTᵣₑₗₑₐₛₑ is greater than 0.001 ml/ms;
k. said volume rate dV_{PF}/dT or dV_{PF}/dTᵣₑₗₑₐₛₑ is greater than 0.01 ml/ms; and
l. any combination thereof.

It is another object of the present invention to provide the method as defined above, additionally comprising at least one of the following steps:
a. selecting said body cavity from a group consisting of a nasal cavity, the mouth, the throat, an ear, the eye, the vagina, the rectum, the urethra, and any combination thereof;
b. selecting said gas from a group consisting of: air, nitrogen, oxygen, carbon dioxide, helium, neon, xenon, nitric oxide and any combination thereof;
c. dispensing said at least one substance, and during said step of dispensing, forming a plume of aerosol with predetermined distribution from a mixture of said predetermined volume V_{gas} [ml] of said pressurized gas and said predetermined volume V_{sub} [ml or mg] entrained within it; selecting said predetermined distribution from a group consisting of: a homogeneous distribution, a heterogeneous distribution; selecting said heterogeneous distribution from a group consisting of: an arbitrary distribution, a distribution in which the density of said at least one substance within said mixture follows a predetermined pattern, and any combination thereof; selecting characteristics of said aerosol from a group consisting of: particle size, particle shape, particle distribution, and any combination thereof, are determinable from characteristics of said device selected from a group consisting of: said predetermined volume of said pressurized gas, said predetermined volume of said substance, said predetermined pressure of said pressurized gas, said predetermined orifice size, and any combination thereof;
d. selecting said substance from a group consisting of: a gas, a liquid, a powder, a slurry, a gel, a suspension, and any combination thereof;
e. storing at least one said substance under either an inert atmosphere or under vacuum, thereby preventing reactions during storage; and
f. characterizing a dose-response curve for brain concentration of said substance to be of substantially linear form; and
g. a dose-response curve for brain concentration having a fit selected from a group consisting of logarithmic, parabolic, exponential, sigmoid, power-low, and any combination thereof of said substance when administered nasally via said device.

It is another object of the present invention to provide the method as defined above, wherein said substance is selected from a group consisting of proteins; stem-cells; cells, organs, portions, extracts, and isolations thereof macro-molecules; RNA or other genes and proteins-encoding materials; neurotransmitters; receptor antagonists; hormones; Ketamine; Baqsimi product commercially available by Lilly (US); Glucagon; substrates to treat one of eth followings: anaphylaxis, Parkinson, seizures and opioid overdose; epinephrine; atropine; metoclopramide; commercially available Naloxone or Narcan products; Esketamine (Spravato); Radicava [edaravone]; Ingrezza [valbenazine]; Austedo [deutetrabenazine]; Ocrevus [ocrelizumab]; Xadago [safinamide]; Spinraza [nusinersen]; Zinbryta [daclizumab]; Nuplazid [pimavanserin]; Aristada [aripiprazole lauroxil]; Vraylar [cariprazine]; Rexulti [brexpiprazole]; Aptiom [eslicarbazepine acetate]; Vizamyl [flutemetamol F18 injection]; Brintellix [vortioxetine]; Tecfidera [dimethyl fumarate]; Dotarem [gadoterate meglumine]; Antibody mediated brain targeting drug delivery including aducanumab, gantenerumab, bapineuzumab, solanezumab, ofatumumab CD20, BIIB033, LCN2, HMGB1; insulin; oxytocin; orexin-A; leptin; benzodiazepine i.e. midazolam; naloxone; perillyl alcohol; camptothecin; phytochemicals including curcumin and chrysin; nucleotides; olanzapine; risperidone; Venlafaxin; GDF-5; zonisamide; ropinirole; plant-originated and synthetically-produced terpenes and cannabinoids, including THC and CBD; valproric acid; rivastigmine; estradiol; topiramate or an equivalent preparation comprising CAS No. 97240-79-4; MFSD2 or MFSD2A or sodium-dependent lysophosphatidylcholine symporter; and any esters, salts, derivatives, mixtures, combinations thereof, with or without a carrier, liposomes, lyophilic or water-miscible solvents, surfactants, cells, cells fractions, at a therapeutically effective concentration.

It is another object of the present invention to provide the method as defined above, wherein said puncturing member comprising a plurality of holes throughout which said pressurized fluid exits said chamber and entrains said substance, after activation of said activation mechanism.

It is another object of the present invention to provide the method as defined above, wherein said capsule is a hollow tube characterized by at least two ends interconnect to each other, at least one of which is positioned proximal to said chamber.

It is another object of the present invention to provide the method as defined above, wherein said capsule comprises at least one spherical element position by at least one of said ends, adapted to seal said capsule and prevent leakage of said at least one substrate therefrom.

It is another object of the present invention to provide the method as defined above, wherein said capsule comprises two spherical elements, each of which is disposed at each of said ends, such that said at least one substrate is positioned therebetween.

It is another object of the present invention to provide the method as defined above, wherein said two spherical elements, once said pressurized fluid exits said chamber, are adapted to mix said at least one substrate and said at least one substrate.

It is another object of the present invention to provide the method as defined above, wherein said capsule comprises at least one membrane positioned by at least one of said ends, adapted to seal said capsule and prevent leakage of said at least one substrate therefrom.

It is another object of the present invention to provide the method as defined above, wherein said capsule comprises two membranes, each of which is disposed at each of said ends, such that said at least one substrate is positioned therebetween.

It is another object of the present invention to provide the method as defined above, wherein said capsule comprises at least one duckbill valve positioned by at least one of said ends, adapted to seal said capsule and prevent leakage of said at least one substrate therefrom.

It is another object of the present invention to provide the method as defined above, wherein said capsule comprises two duckbill valves, each of which is disposed at each of said ends, such that said at least one substrate is positioned therebetween.

It is another object of the present invention to provide the method as defined above, wherein said capsule comprises at least one spherical element, membrane, duckbill valve and any combination thereof.

It is another object of the present invention to provide the method as defined above, wherein said at least one hollow puncturing member comprises at least one orifice throughout which said pressurized fluid enters said hollow puncturing member.

It is another object of the present invention to provide the method as defined above, wherein, once said device is actuated, said pressurized fluid exit said chamber and enters said hollow puncturing member through said at least one orifice.

It is another object of the present invention to provide the method as defined above, wherein said at least one hollow puncturing member comprises at least one orifice throughout which said pressurized fluid enters said hollow puncturing member.

It is another object of the present invention to provide the method as defined above, wherein, once said device is actuated, said pressurized fluid exit said chamber and enters said hollow puncturing member through said at least one orifice.

It is an object of the present invention to disclose a device for delivering either one or more substances within at least one body cavity. The device is characterized by at least one pierceable vial comprising V_{sub} [ml or mg] of the substances; the vial having at least one fluid inlet port of diameter Dᵢₙ [mm] and at least one fluid discharging outlet port of diameter Dₒᵤₜ [mm], configured for placement in proximity to the body cavity; the fluid inlet port configured by means of size and shape to interface with at least one puncturing member, configured to, upon coupling to the fluid inlet port, piercing the same, thereby providing the substances in a fluid communication, with at least one chamber configured to accept pressurized fluid at volume V_{PF} [ml] and pressure P_{PF} [barg]; the pressurized fluid flows from the chamber, via the fluid inlet port, entrains the substances, erupts via the fluid discharging outlet port to within the body cavity in the form of aerosol, such that the release time of the V_{sub} [ml or mg] of the substances and the V_{PF} [ml] of the pressurized fluid, dTᵣₑₗₑₐₛₑ is less than 500 milliseconds; In this device, the following held: V_{PF} is in a range of 1 to 50 ml; V_{sub} is in a range of about 0.01 to about 7 ml or 0.1 mg to 7 g; P_{PF} is in a range of about 0 to about 10 barg; further wherein at least one of the following is being held true: Dᵢₙ or Dₒᵤₜ are in a range of 0.2 to 6 mm; the pressure velocity is greater than 0.001 barg/ms; the pressure velocity is greater than 0.01 barg/ms; the volume rate dV_{sub}/dTᵣₑₗₑₐₛₑ is greater than 0.0001 ml/ms; the volume rate dV_{sub}/dTᵣₑₗₑₐₛₑ is greater than 0.001 ml/ms; the volume rate dV_{PF}/dTᵣₑₗₑₐₛₑ is greater than 0.001 ml/ms; the volume rate dV_{PF}/dTᵣₑₗₑₐₛₑ is greater than 0.01 ml/ms; any combination thereof. The vial is further selected from a group consisting of a pierceable container, a blow-fill-seal and a form-fill-seal and any combination thereof.

It is another of the present invention to disclose the device as disclosed above, wherein the vial comprises a cap adapted to seal the vial, such that removal thereof provides fluid communication between the vial and the body cavity through the fluid discharging outlet port.

It is another of the present invention to disclose the device as disclosed in any of the above, wherein the at least one puncturing member is adapted to pierce the vial be means of a screw mechanism, such that rotation of the nosepiece cover along the screw mechanism in the base enables the pierce of the fluid inlet port in the vial by means of the puncturing member.

It is another of the present invention to disclose the device as defined in any of the above, wherein at least one of the following is true: (a) The body cavity is selected from a group consisting of nasal cavity, the mouth, the throat, an ear, the eye, the vagina, the rectum, the urethra, and any combination thereof. (b) The pressurized gas is selected from a group consisting of air, nitrogen, oxygen, carbon dioxide, helium, neon, xenon, nitric oxide and any combination thereof. (c) During dispensing of the at least one substance, a mixture of the predetermined volume V_{gas} [ml] of the pressurized gas with the predetermined volume V_{sub} [ml or mg] of the substance entrained within it forms a plume of aerosol; the aerosol having a predetermined distribution, the distribution being either homogeneous or heterogeneous, the heterogeneous distribution is selected from a group consisting of: an arbitrary distribution, a distribution in which the density of the at least one substance within the mixture follows a predetermined pattern, and any combination thereof; characteristics of the aerosol selected from a group consisting of: particle size, particle shape, particle distribution, and any combination thereof, are determinable from characteristics of the device selected from a group consisting of: the predetermined volume of the pressurized gas, the predetermined volume of the substance, the predetermined pressure of the pressurized gas, the predetermined orifice size, and any combination thereof. (d) At least one the substance is selected from a group consisting of a gas, a liquid, a powder, an aerosol, a slurry, a gel, a suspension and any combination thereof. (e) The least one the substance is stored under either an inert atmosphere or under vacuum to prevent reactions during storage. (f) A dose-response curve is substantially linear for brain concentration of the substance when administered nasally via the device; and (g) A dose-response curve for brain concentration having a fit selected from a group consisting of logarithmic, parabolic, exponential, sigmoid, power-low, and any combination thereof; of the substance when administered nasally via the device.

It is another of the present invention to disclose the device as disclosed in any of the above, wherein the vial is a capsule having a main longitudinal axis, the capsule comprising a number n of compartments, the capsule configured to contain the predetermined volume V_{sub} [ml or mg] of the at least one substance, the volume V_{sub} [ml or mg] of the at least one substance containable in at least one of the n compartments; at least one of the following being true: the number n of the compartments is an integer greater than or equal to 1; at least one the compartment has cross-section with shape selected from a group consisting of: wedge shaped, circular, oval, elliptical, polygonal, annular, and any combination thereof; for the number n of compartments being an integer greater than 1, at least two the compartments have different volumes; for the number n of compartments being an integer greater than 1, at least two the compartments have the same volume; for the number n of compartments being an integer greater than 1, at least two the compartments have different cross-sectional areas; for the number n of compartments being an integer greater than 1, at least two the compartments have the same cross-sectional area; for the number n of compartments being an integer greater than 1, at least two the compartments contain different substances; for the number n of compartments being an integer greater than 1, at least two the compartments contain the same substance; for the number n of compartments being an integer greater than 1, at least two the compartments are disposed coaxially around the main longitudinal axis of the capsule; for the number n of compartments being an integer greater than 1, at least two the compartments are disposed sequentially along the main longitudinal axis of the capsule; for the number n of compartments greater than 1, the plurality of substances mix during the dispensing; and for the number n of compartments greater than 1, the plurality of substances react during the dispensing.

It is another of the present invention to disclose the device as disclosed in any of the above, wherein the vial comprises a port fluidly connectable to the exterior of the device, the port configured such that the at least one substance is insertable into the chamber via the port.

It is another object of the present invention to disclose the device as disclosed above, wherein the device comprises a port cover configured to provide an air-tight closure for the port, the port cover slidable along the device, rotatable around the device, rotatable around a hinge on the exterior of the device and any combination thereof.

It is another of the present invention to disclose the device as disclosed in any of the above, wherein the pressurized fluid entrains the substance in a pulsed manner, such that a plurality of portions V_{PF} erupts via the fluid discharging outlet to within the body cavity

It is another object of the present invention to disclose a method for delivering either one or more substances within at least one body cavity, characterized by steps of providing at least one pierceable vial with V_{sub} [ml or mg] of the substances; the vial having at least one fluid inlet port of diameter Dᵢₙ [mm] and at least one fluid discharging outlet port of diameter Dₒᵤₜ [mm], configured for placement in proximity to the body cavity; configuring the fluid inlet by means of size and shape to interface a puncturing member, so that upon coupling to the fluid inlet port, piercing of the same, thereby providing the substances in a fluid communication, with at least one chamber configured to accept pressurized fluid at volume V_{PF} [ml] and pressure P_{PF} [barg]; and

facilitating the flow of the pressurized fluid from the chamber, via the fluid inlet, entrains the substances, erupts via the fluid discharging outlet port into the body cavity, such that the release time of the V_{sub} [ml or mg] of the substances and the V_{PF} [ml] of the pressurized fluid, dTᵣₑₗₑₐₛₑ is less than 500 milliseconds. The method further held the followings: V_{PF} is in a range of 1 to 50 ml; V_{sub} is in a range of about 0.01 to about 7 ml or 0.1 mg to 1 g; P_{PF} is in a range of about 0 to about 10 barg/The following is further being held true: Dᵢₙ or Dₒᵤₜ are in a range of 0.2 to 6 mm; the pressure velocity is greater than 0.001 barg/ms; the pressure velocity is greater than 0.01 barg/ms; the volume rate or dV_{sub}/dTᵣₑₗₑₐₛₑ is greater than 0.0001 ml/ms; the volume rate dV_{sub}/dTᵣₑₗₑₐₛₑ is greater than 0.001 ml/ms; the volume rate dV_{PF}/dTᵣₑₗₑₐₛₑ is greater than 0.001 ml/ms; the volume rate dV_{PF}/dTᵣₑₗₑₐₛₑ is greater than 0.01 ml/ms; and any combination thereof. Additionally, the step of providing the vial additionally comprising step of selecting the vial from a group consisting of a pierceable container, a blow-fill-seal and a form-fill-seal and any combination thereof.

It is another of the present invention to disclose the method as disclosed above, wherein it additionally comprising at least one of the following steps: selecting the body cavity from a group consisting of a nasal cavity, the mouth, the throat, an ear, the eye, the vagina, the rectum, the urethra, and any combination thereof; selecting the gas from a group consisting of: air, nitrogen, oxygen, carbon dioxide, helium, neon, xenon, nitric oxide and any combination thereof; dispensing the at least one substance, and during the step of dispensing, forming a plume of aerosol with predetermined distribution from a mixture of the predetermined volume V_{gas} [ml] of the pressurized gas and the predetermined volume V_{sub} [ml] entrained within it; selecting the predetermined distribution from a group consisting of: a homogeneous distribution, a heterogeneous distribution; selecting the heterogeneous distribution from a group consisting of: an arbitrary distribution, a distribution in which the density of the at least one substance within the mixture follows a predetermined pattern, and any combination thereof; selecting characteristics of the aerosol from a group consisting of: particle size, particle shape, particle distribution, and any combination thereof, are determinable from characteristics of the device selected from a group consisting of: the predetermined volume of the pressurized gas, the predetermined volume of the substance, the predetermined pressure of the pressurized gas, the predetermined orifice size, and any combination thereof;

selecting the substance from a group consisting of: a gas, a liquid, a powder, a slurry, a gel, a suspension, and any combination thereof; storing at least one the substance under either an inert atmosphere or under vacuum, thereby preventing reactions during storage; and characterizing a dose-response curve for brain concentration of the substance to be of substantially linear form; and a dose-response curve for brain concentration having a fit selected from a group consisting of logarithmic, parabolic, exponential, sigmoid, power-low, and any combination thereof; of the substance when administered nasally via the device.

### BRIEF DESCRIPTION OF THE DRAWINGS

The invention is herein described, by way of example only, with reference to the accompanying drawings, wherein:

FIGS. 1A-B shows an embodiment of the present invention, with FIG. 1A showing an exploded view of the device and FIG. 1B showing the device fully assembled;

FIGS. 2A-C shows another embodiment of the present invention, with FIG. 2A showing the device, FIG. 2B showing a cross section of the device and FIG. 2C showing an enlarged view of a portion of the device;

FIGS. 3A-D shows another embodiment of the present invention, with FIG. 3A showing a cross-section of the device, FIG. 3B showing an enlarged view a portion of the device, FIG. 3C showing the exterior of the nosepiece and FIG. 3D showing the device from the top;

FIGS. 4A-C shows another embodiment of the present invention, after activation, with the device shown in FIG. 4A, a cross section of the device in FIG. 4B and an enlarged view a portion of the device in FIG. 4C;

FIGS. 5A-G show another embodiment of the device, with FIG. 5A showing a side view of a pre-used device carrying a BFS, FIG. 5B showing a cross section of the same, FIGS. 5C and 5D depicting the device when connected to a BFS, FIG. 5E showing the same when the device is ready to use, FIG. 5F showing connection of the BFS to the device; and FIG. 5G showing the device after activation;

FIGS. 6A-E show another embodiment of the present invention, with FIG. 6A illustrating a front view of a pre-used device carrying a BFS and cross sections of the same; FIG. 6B showing securing of the BFS to the device and breaking the cap; FIG. 6C presenting a cross-section view; FIG. 6D showing a button at the base of the device being pushed; FIG. 6E showing pressurized fluid flowing from the container to the BFS and carrying the drug outward;

FIGS. 7A-7B illustrating a device being another embodiment of the present invention, a front and exploded view, respectively;

FIGS. 8A-8E show same device in various modes of operation; and

FIG. 9 depict two views of the device interconnected to a safety latch according to an embodiment of the present invention.

FIGS. 10-19 show another embodiment of the present invention.

FIGS. 20a-20d illustrating different embodiments of the hollow needle.

FIG. 21 shows another embodiment of the present invention.

### DETAILED DESCRIPTION OF THE PREFERRED EMBODIMENTS

The following description is provided, alongside all chapters of the present invention, so as to enable any person skilled in the art to make use of the invention and sets forth the best modes contemplated by the inventor of carrying out this invention. Various modifications, however, will remain apparent to those skilled in the art, since the generic principles of the present invention have been defined specifically to provide a device capable of improving the transfer of medicament to a predetermined desired location and to provide a device capable of improving the delivery of medicament through the tissue.

In the present invention, a combination of parameters and forces such as pressure, gas/air volume orifice diameter enable the formation of optimized aerosol characteristics for both improved delivery of aerosol to the target area (such as the olfactory epithelium in the nasal cavity) and enhanced absorption at that area for better delivery to a desired tissue (such as the brain).

The term ‘ul’ or ‘μm’ hereinafter refers to the unit micro liters or micro meters, respectively.

The term ‘capsule’ interchangeably ‘container’ interchangeably refer to a container configured to contain a flowable substance. The term flowable refers hereinafter to any liquid, gas, aerosol, powder and any combination thereof. It should be emphasized that the term capsule can also refer to a predefined volume within the same in which a flowable substance is placed. In other words, the predefined volume is sized and shaped to enclose a predefined volume of the sub stance.

The term ‘high barrier films’ hereinafter refers to any materials in flexible packaging laminations that prevent the permeation of water, water vapor, oil, oxygen, aroma, flavor, gas, or light. Such provision is enabled by low permeability of the film. In some embodiments the film is made of high-density Aluminum films. Thus, according to one embodiment of the present invention, the pressurized gas enclosed within high barrier film that can maintain the pressure of the compressed gas for prolong period of times without leakage therefrom.

The term ‘plurality’ hereinafter refers to an integer greater than or equal to one.

The term ‘olfactory epithelium’ hereinafter refers to a specialized epithelial tissue inside the nasal cavity. The olfactory epithelium lies in the upper top portion of the nasal cavity.

The term ‘substance’ hereinafter refers to any substance capable of flowing. Such a substance can be a granular material, including a powder; a liquid; a gel; a slurry; a suspension; and any combination thereof. The term further refers to one or more members of a group consisting of proteins; stem-cells; cells, organs, portions, extracts, and isolations thereof; macro-molecules; RNA or other genes and proteins-encoding materials; neurotransmitters; receptor antagonists; biologic response modifiers; hormones; Ketamine; commercially available by Lilly (US) Baqsimi product; Glucagon, biologic response modifiers; Glucagon; substrates to treat one of eth followings: anaphylaxis, Parkinson, seizures and opioid overdose; epinephrine; atropine; metoclopramide; commercially available Naloxone or Narcan products; Esketamine (Spravato); Radicava [edaravone]; Ingrezza [valbenazine]; Austedo [deutetrabenazine]; Ocrevus [ocrelizumab]; Xadago [safinamide]; Spinraza [nusinersen]; Zinbryta [daclizumab]; Nuplazid [pimavanserin]; Aristada [aripiprazole lauroxil]; Vraylar [cariprazine]; Rexulti [brexpiprazole]; Aptiom [eslicarbazepine acetate]; Vizamyl [flutemetamol F18 injection]; Brintellix [vortioxetine]; Tecfidera [dimethyl fumarate]; Dotarem [gadoterate meglumine]; Antibody mediated brain targeting drug delivery including aducanumab, gantenerumab, bapineuzumab, solanezumab, ofatumumab CD20, BIIB033, LCN2, HMGB1; insulin; oxytocin; orexin-A; leptin; benzodiazepine i.e. midazolam; naloxone; perillyl alcohol; camptothecin; phytochemicals including curcumin and chrysin; nucleotides; olanzapine; risperidone; Venlafaxin; GDF-5; zonisamide; ropinirole; plant-originated and synthetically-produced terpenes and cannabinoids, including THC and CBD; valproric acid; rivastigmine; estradiol; topiramate or an equivalent preparation comprising CAS No. 97240-79-4; MFSD2 or MFSD2A or sodium-dependent lysophosphatidylcholine symporter; and any esters, salts, derivatives, mixtures, combinations thereof, with or without a carrier, liposomes, lyophilic or water-miscible solvents, surfactants, cells, cells fractions, at a therapeutically effective concentration.

The term ‘gas’ refers to any fluid that can be readily compressed. Gases as used herein include, but are not limited to, air, nitrogen, oxygen, carbon dioxide, helium, neon, xenon, nitric oxide and any combination thereof. Devices charged by hand will typically use air as the carrier gas.

The term ‘fluid’ refers to any substance or mixtures of substances that continually deforms (flows) under an applied shear stress, or external force. This term refers to gas, liquids, particulate or granulated solids (powders), aerosols, and any mixtures and combinations thereof.

The term ‘about’ refers hereinafter to a range of 25% below or above the referred value.

The term “body orifice” and “body cavity” are interchangeably refer to one or more of the followings: nasal cavity, a mouth, a throat, an ear, a vagina, a rectum, a urethra, and any combination thereof.

The term ‘biologic’ or ‘biologic response modifier’ hereinafter refers to material manufactured in or extracted from biological sources such as a genetically engineered protein derived from human genes, or a biologically effective combination of such proteins.

All pressures herein are gauge pressures, relative to atmospheric pressure. Pressure units will be written herein using the standard abbreviation for “gauge”, namely, “g”. For example, atmospheric pressure is 0 barg and a pressure of 1 bar above atmospheric is 1 barg.

The term ‘release time’ refers hereinafter to the time for the drug and carrier gas to substantially completely exit the device. Typically, the release time is affected by the combination of the Volume of substance, volume of pressurized gas, pressure of pressurized gas, the orifice diameter, the activation time of the valve that reflects the time for the device to reconfigure from the ACTIVE configuration to the INACTIVE configuration or vice versa and any combination thereof.

The terms ‘the device’, ‘the present device’, ‘the SipNose device’ and ‘SipNose’ will be used interchangeably to refer to a device according to any embodiment of the present invention.

In all of the embodiments of the device shown hereinbelow, identical numbers refer to identical functions. All figures shown herein are illustrative and none is to scale.

The present invention teaches a device for delivering a predetermined amount of a substance, preferably comprising a medication or combination of medications, into a body orifice of a subject, the orifice comprising any of the body's natural orifices, including a nostril, the mouth, the ear, the throat, the urethra, the eye, the vagina, the rectum and any combination thereof.

In preferred embodiments of the device, the device comprises a delivery mechanism and a medicament capsule, as described hereinbelow. The device can apply a broad range of drugs and materials to the nasal cavity for local effect, deliver a broad range of drugs and materials through the nasal cavity to the systemic circulation, deliver a broad range of drugs and materials through the nasal cavity to the central nerve system (CNS) the brain, spinal cord and associated nerves, and any combination thereof.

The drugs to be applied could be, but are not limited to, pharmaceuticals, natural compounds, biologics, hormones, peptides, proteins, viruses, cells, stem cells and any combination thereof.

However, it should be emphasized that the device can be provided alone as well as in combination with a capsule.

In some cases, the capsule would be provided with a known medicament within the same and in other cases the capsule would be ‘filled’ with the medicament just before use.

In some embodiments of the present invention, the device operating characteristics and the substance characteristics can be jointly optimized to maximize uptake of the substance at the desired site. In preferred variants of such embodiments, uptake is further optimized by exploiting synergies between delivery characteristics generated by the device and by the formulation or composition of the delivered material

In some embodiments, the substance comprises one or more agents to optimize delivery through the mucosal membrane by means of mucoadhesive agent and/or a permeability enhancer agent and/or a particulate formulation in the nano-particle or macro-particle range, and any combination thereof. In such embodiments, the combination of the device and substance enhance the delivery of the active agent to the target area (nasal epithelium and more specifically olfactory epithelium) and from there to the target tissue (for example the brain).

A non-limiting example is a composition comprising a drug to be delivered and at least one chemical permeation enhancer (CPE). In a preferred embodiment, the composition contains two or more CPEs which, by using a nasal delivery device, affect in an additive manner or behave synergistically to increase the permeability of the epithelium, while providing an acceptably low level of cytotoxicity to the cells. The concentration of the one or more CPEs is selected to provide the greatest amount of overall potential (OP). Additionally, the CPEs are selected based on the treatment. CPEs that behave primarily by transcellular transport are preferred for delivering drugs into epithelial cells. CPEs that behave primarily by paracellular transport are preferred for delivering drugs through epithelial cells. Also provided herein are mucoadhesive agents that enable the extension of the exposure period of the target tissue/mucus membrane to the active agent, for the enhancement of delivery of the active agent to and through the mucus membrane.

In contrast to prior-art nasal delivery devices and technologies, the devices of the present invention can produce a fine aerosol in the nasal cavity or other desired body orifice at the target area and at the location of the target tissue instead of producing the aerosol only within the device or immediately after exit from the device. Utilizing the pressure as a driving force and the air as a carrier allows the material to be released from the nozzle as a mixture of aerosol and a pre-aerosolized state. The properties of the resultant aerosol are typically dependent on the properties of the device and of the medium into which the device is discharged. The properties of the device which affect the aerosol characteristics are the delivery pressure, the volume of the delivery gas, the characteristics of its orifice and time of activate.

In some embodiments, the aerosol properties are fairly independent of the delivered substance, while, in other embodiments, the pressure, volume, orifice characteristics, and delivered substance properties can be co-optimized.

In prior-art devices the aerosol is produced in proximity exit of the device. Typically, the aerosol comprises a wide “fan” of aerosol and a low driving force. Therefore, large droplets typically deposit very close to the exit from the device, while smaller droplets tend to quickly contact the walls of the passage, so that deposition is typically predominantly close to the delivery end of the device, with little of the substance reaching desired sites deeper in the body orifice, such as the middle and superior turbinates of the nose. In the present invention the aerosol created, due to the pressurized air carrier, reaches the upper regions of the nasal cavity.

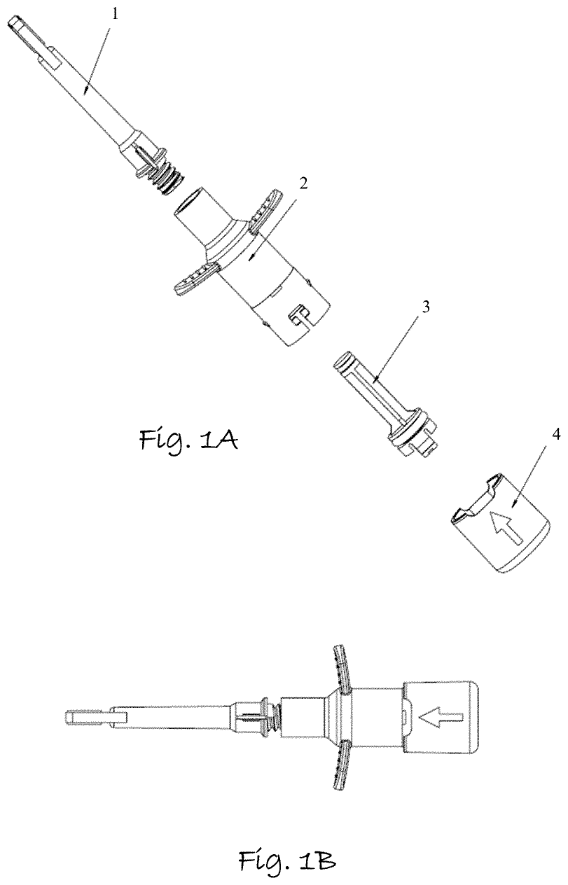

Reference is now made to FIG. 1A-B, disclosing a device according to one embodiment of the present invention. FIG. 1A shows an exploded view of the device, while FIG. 1B shows the device fully assembled. As shown in FIG. 1A, the device comprises, inter alia, a BFS nose piece (1), a pressurized-fluid container (2), an air chamber gate (3) and an activation mechanism base (4).

In the FIG. 1A, the base (4), an air chamber gate (3) has with a first gate O-ring at its proximal end and a second gate O-ring at its distal end (both shown in FIGS. 7A-7B and 8A-8E, as numerical reference 77 and 78).

The pressurized-fluid container (2) will fit over the air chamber gate (3), with the first gate O-ring and the second gate O-ring providing airtight seals before activation so that compressed gas is storable between the air chamber gate (3) and the pressurized-fluid container (2).

As will be disclosed hereinafter, the pierceable drug container (1) (e.g., BFS) in the nosepiece, where there is a puncturing element that punctures the drug container and once the compressed gas is released from the pressurized-fluid container (2), the same entrains the drug and deliver the same to the nasal cavity (see FIGS. 7A-7B and 8A-8E).

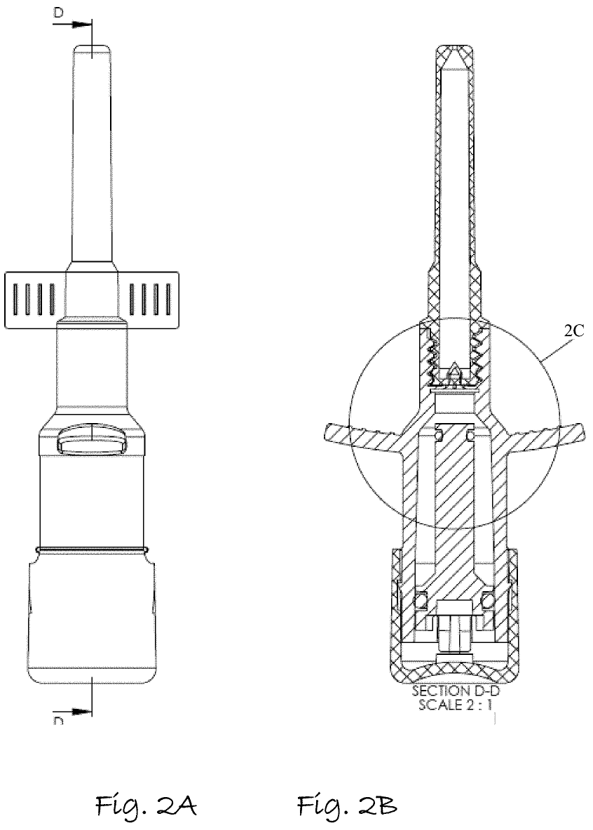

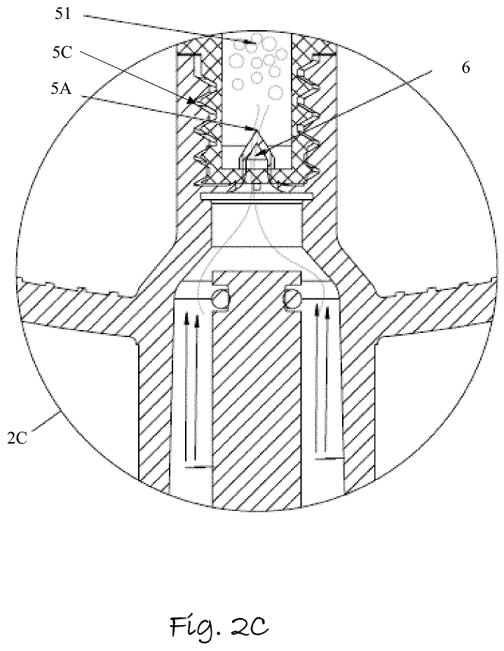

As shown in the Figs. the base of the device forms the activation button (4); to activate, the activation button (4) is pressed upward, then the air chamber gate (3) is drawn downwardly, which removes the sealing of the upper O-ring (78 in FIGS. 7A-7B). The movement of the air chamber gate (3) opens a gap between the pressurized-fluid container (2) and the BFF nose piece (1), allowing the pressurized-fluid to escape from container 2, enter BFF nose piece (1) (after the same has been pierced by the piercing needle 79, shown in FIGS. 7A-7B), and entrain the substance to the nasal cavity. Reference is now made to FIGS. 2A-C, disclosing a device according to another embodiment of the present invention. FIG. 2B depicts a cross section along the line D:D of the device as shown in FIG. 2A. The area within the circle 2C in FIG. 2B is shown enlarged in FIG. 2C, where the device's spike is disclosed (6). Also seen in FIG. 2C is a BSF lower BFS point at which the needle punctures the BFS(5A), BSF nosepiece which contain the drug (51) and an activation screw mechanism (5C).

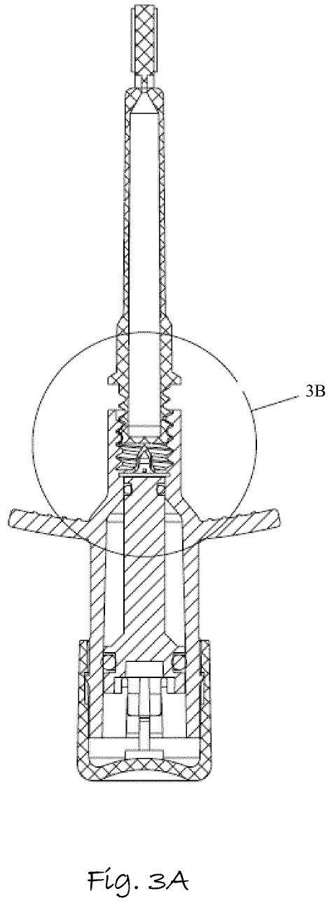

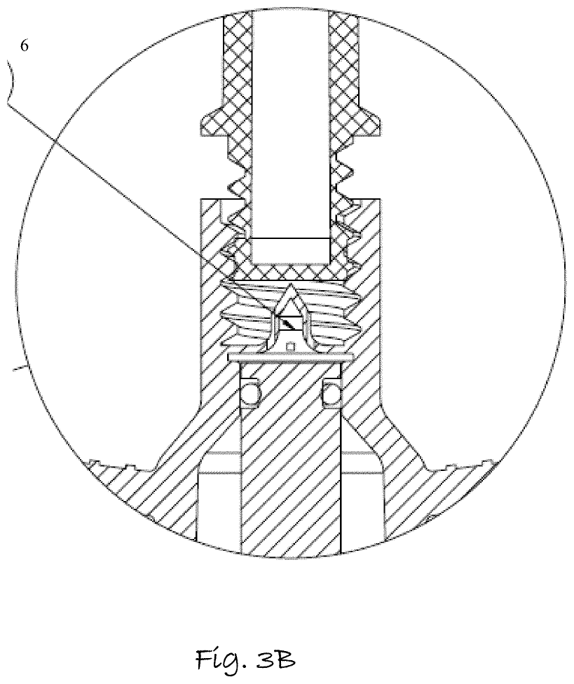

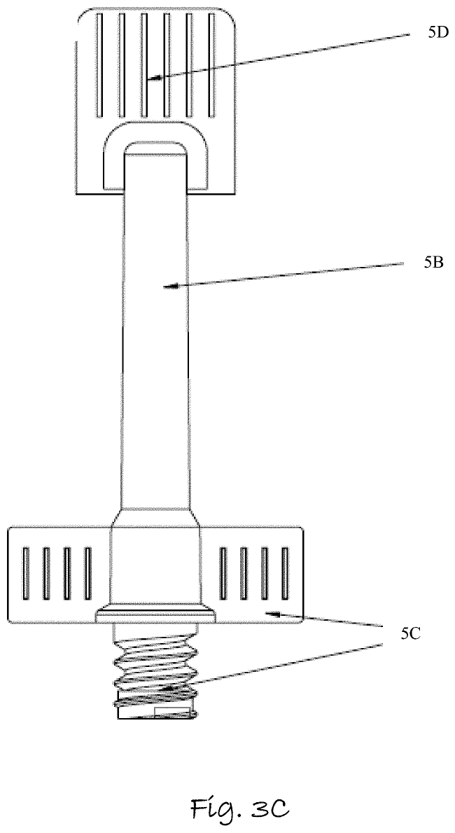

Reference is now made to FIGS. 3A-D, disclosing a device according to another embodiment of the present invention. FIG. 3A shows a cross-section of the device. FIG. 3B shows an enlarged view of the area inside the circle 3B of FIG. 3A. The piercing member (6) can be clearly seen. FIG. 3C shows the exterior of the nosepiece, showing the activation screw mechanism (5C) that is tightened in order to drive the bottom of the drug container against the spike and thereby pierce the drug container; the nosepiece cover (5D) and the main body of the nosepiece (5B). FIG. 3D shows the device from the top.

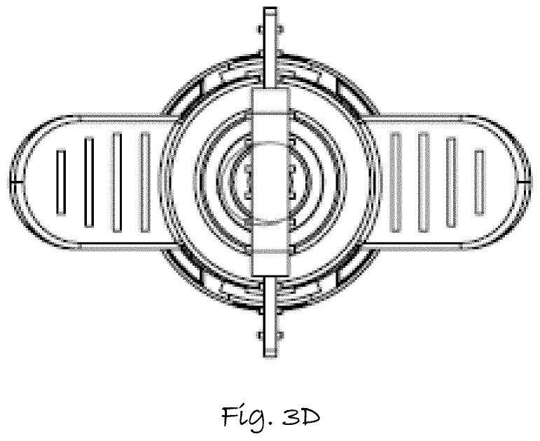

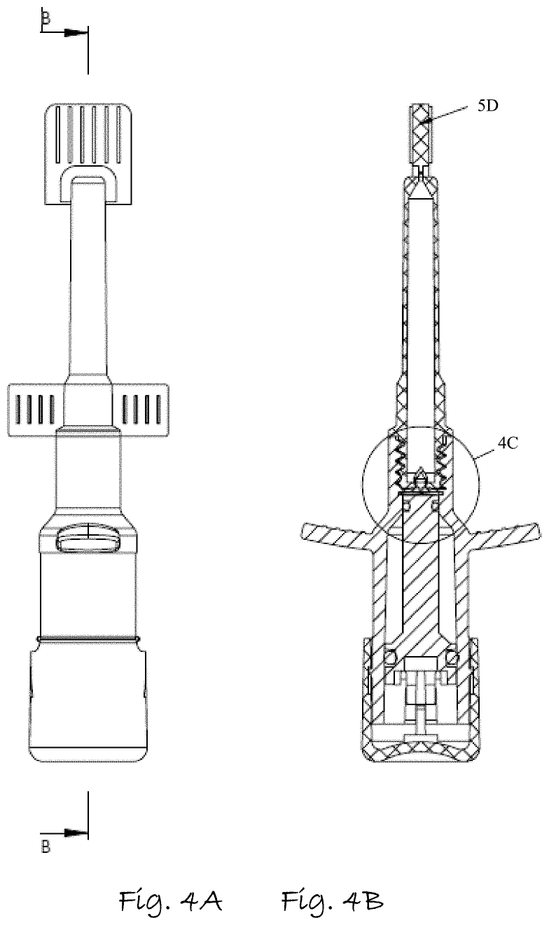

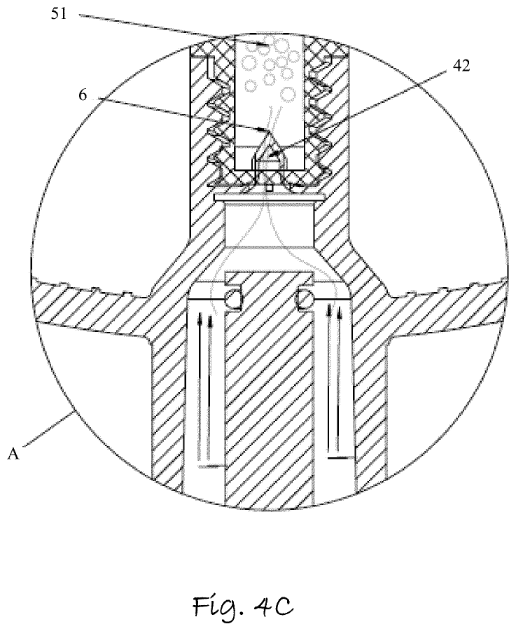

Reference is now made to FIGS. 4A-C, disclosing a device according to another embodiment of the present invention. Here a device is after the activation (FIG. 4A). FIG. 4B shows a cross section of the same along the line B:B. The area within circle 4C is shown enlarged in FIG. 4C, namely a cross section of the piercing member. Drug powder and/or liquid schematically illustrated (51). Air flow through the spike holes (42) entrains the drug.

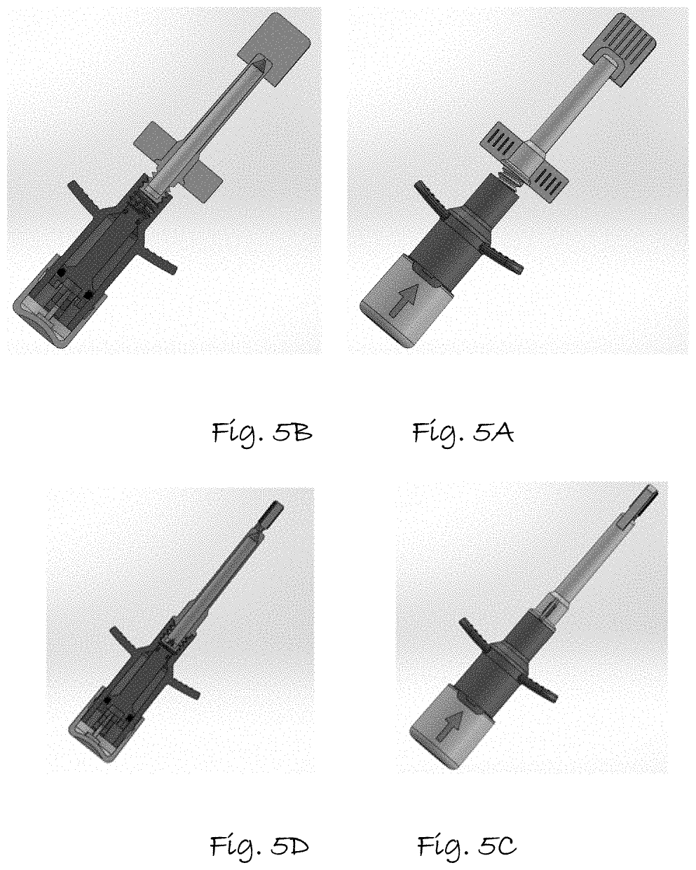

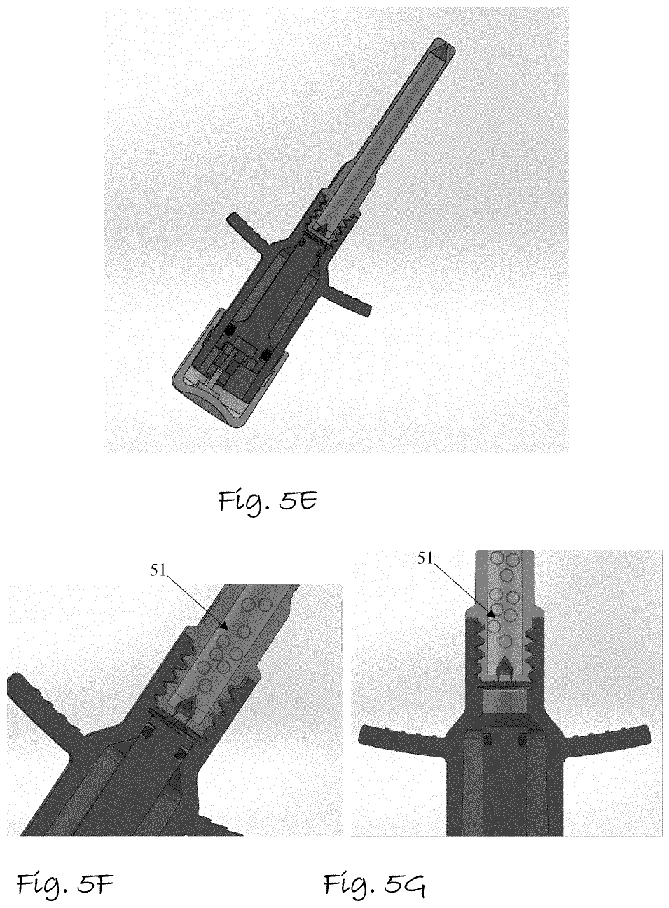

Reference is now made to FIGS. 5A-G, disclosing a device according to another embodiment of the present invention. FIG. 5A is a side view of a pre-used device carrying a BFS, and FIG. 5B is a cross section of the same. FIGS. 5C and 5D similarly depict the device when connected to a BFS. FIG. 5E shows the same when the device is ready to use, FIG. 5F illustrates the connection between the BFS to the device; drug (51) is shown. The device after activation presents the flowing drug (51) in FIG. 5G.

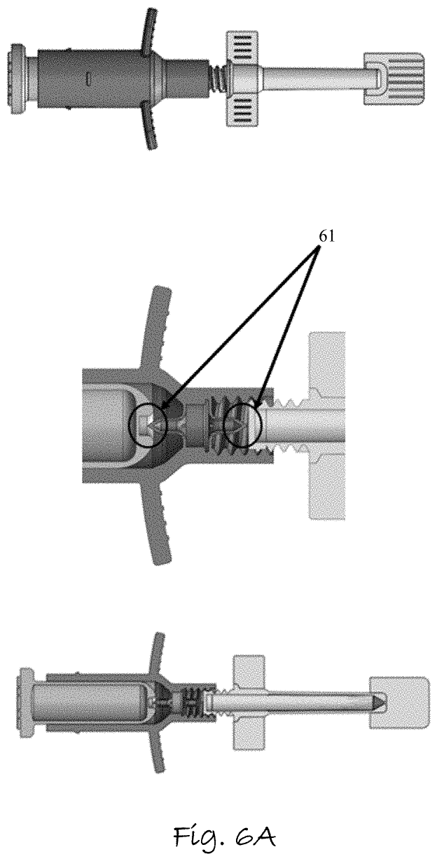

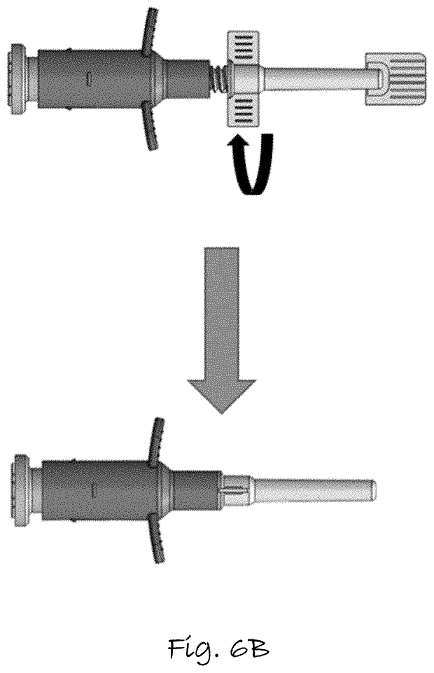

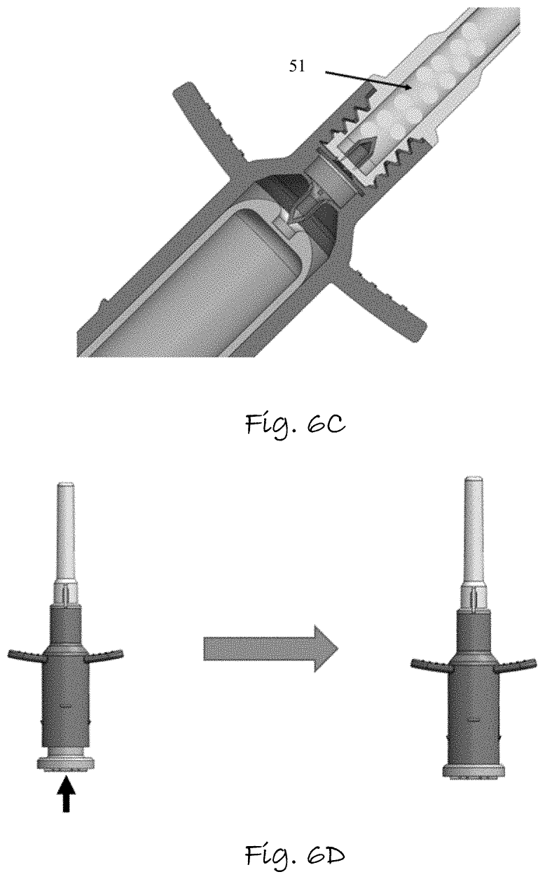

Reference is now made to FIGS. 6A-E disclosing a device according to another embodiment of the present invention. FIG. 6A illustrates a side view (image on the top) of a pre-used device carrying a BFS. Images on the middle and in the bottom are cross sections of the same, showing BFS nosepiece and BFS air container before contact. FIG. 6B shows the second step after introducing the BFS, namely securing the BFS to the device, here by turning the nosepiece of the BFS clockwise. Upon rotation of the nosepiece, the piercing member 511 (shown in FIG. 6C), pierces the drug compartment. A further step is removing (e.g., breaking) the cap, the image at the bottom presents the device after breaking the said cap. The drug (51) is presented in cross section view of FIG. 6C. In FIG. 6D, a button at the base of the device is pushed. Such push actuates the base and a second piercing member 611 pierces the container 80. Subsequently, as depicted in FIG. 6E, pressurized fluid (air, nitrogen etc.) flows from its container (62) to the drug-containing BFS and carries the drug (liquid phase, solid powder particles etc.) (51) outwardly.

One should also note that this example is shown for the same invention but with another kind of pressurized gas container and a different way of compressed gas discharge (by puncturing the container rather than the gate that is shown in the previous figures.

Reference is now made to FIG. 7A, FIG. 7B and FIGS. 8A-8E disclosing a device according to another embodiment of the present invention in a side view and exploded view, respectfully; wherein 70 is a cover holding area; 72 is a pressurized fluid container; 73 is an activation mechanism base; 74 is a cover's body; 75 is a nosepiece; 76 is an air chamber gate; 78 and 77 are O-rings; 79 is a needle; 75 is a nosepiece one way screwing mechanism; 710 is a drug's space; 712 is an air chamber gate's legs; 713 is an air chamber gate's snaps; 714 is a drug storage container locking notch; 715 is a drug storage container locking pin; 716 is an orifice-creating piercing needle; 717 is an orifice; 718 is an aerosol; 720 is a safety latch; 721 and 722 locks; 723 is a pressurized fluid container's internal screwing mechanism. The device comprises modules 70-79 and 710-720, where 71, 74, and 713, and nozzle (orifice) 717 are related with the nosepiece; 72, 76-79, 710-712, 716, to the body and module 73 and 713 is in device's operating button.

In the following sets of figures, namely FIG. 8A-8E, operation modes are illustrated, illustrating a method for delivering either one or more substances within at least one body cavity, characterized by steps of providing a vial with V_{sub} [ml] of said substances; said vial selected from a pierceable container, a blow-fill-seal and a form-fill-seal, further providing said vial with a fluid inlet and a fluid discharging outlet of diameter D [mm], configured for placement in proximity to said body cavity; configuring said fluid inlet by means of size and shape to interface a puncturing member, so that upon coupling to the fluid inlet, piercing of the same, thereby providing the substances in a fluid communication, via a valve, with a chamber configured to accept pressurized fluid at volume V_{PF} [ml] (or mg) and pressure P_{PF} [barg]; the valve is commutable from an CLOSE to an OPEN CONFIGURATION within a short period of time, <500 milliseconds (dT); at the OPEN CONFIGURATION, facilitating the flow of the pressurized fluid to from the chamber, via the fluid inlet, entraining the substances; and then erupting via the fluid discharging outlet to within the body cavity, such that the release time of the V_{sub} [ml or mg] of the substances and the V_{PF} [ml] of the pressurized fluid, dTᵣₑₗₑₐₛₑ is less than 500 milliseconds. Each pf the figures comprises a front view (left side), cross-section (A:A, middle) and isometric view (right side). FIGS. 8B and 8C further depict a rotation mechanism 714-715, which allows a rotation (here, ¼ rotation) thereby enabled the piercing of the nosepiece substance container.

According to another embodiment, the rotation results in a double piercing of the nosepiece substance container and the pressurized air container.

According to another embodiment the pressurized air container is sealed by means of at least one O-ring, such that movement of the o-ring removes the sealing and enables the release of the pressurized air. In some embodiment at least 2 o-rings are used. One o-ring at the bottom of the pressurized air container and the second at the upper portion of the pressurized air container to seal and separate between the pressurized air container and the nosepiece substance container.

At final step (FIG. 8E), upon pressing the activation mechanism base 73, results in movement of the air chamber gate 76 and the upper o-ring to thereby enable the release of the pressurized air from the pressurized air container and into the nosepiece substance container to entrain the same. Once the pressurized air entrains the substance, aerosol 718 is provided throughout the orifice 717, having a narrow plume angle (θ). It is in the scope of the invention wherein the cover comprises means to protect the drug from UV, e.g., photoprotective agents, such as oxybenzone, titanium oxide and octyl methoxycinnamate.

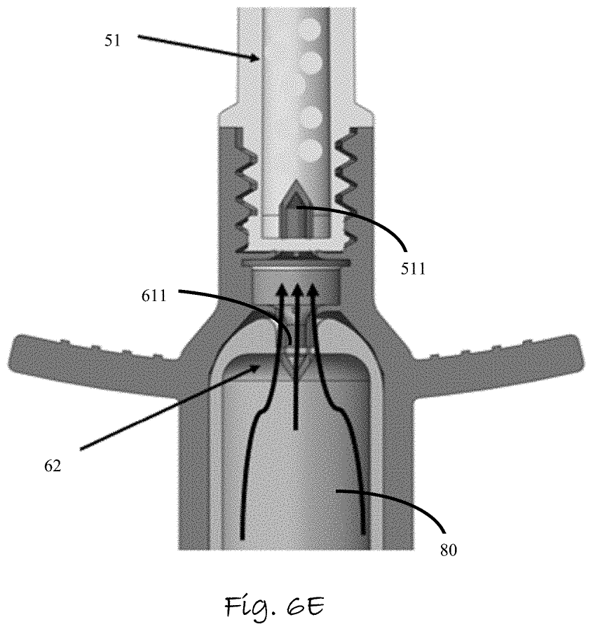

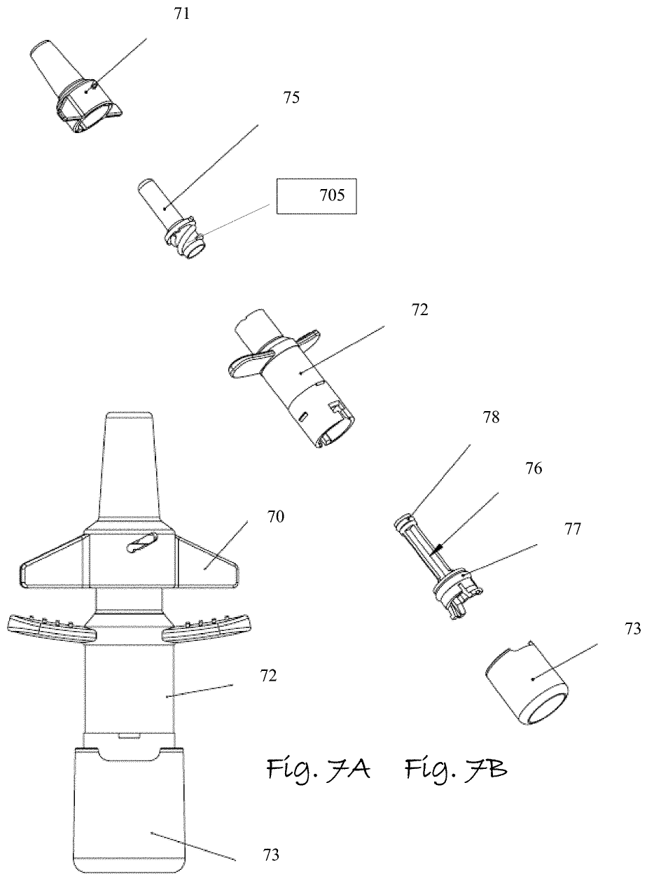

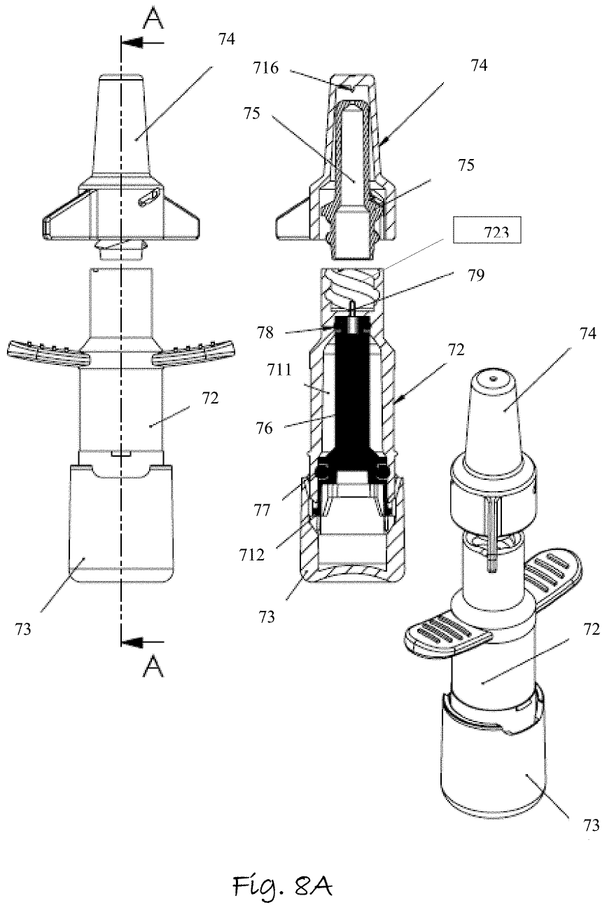

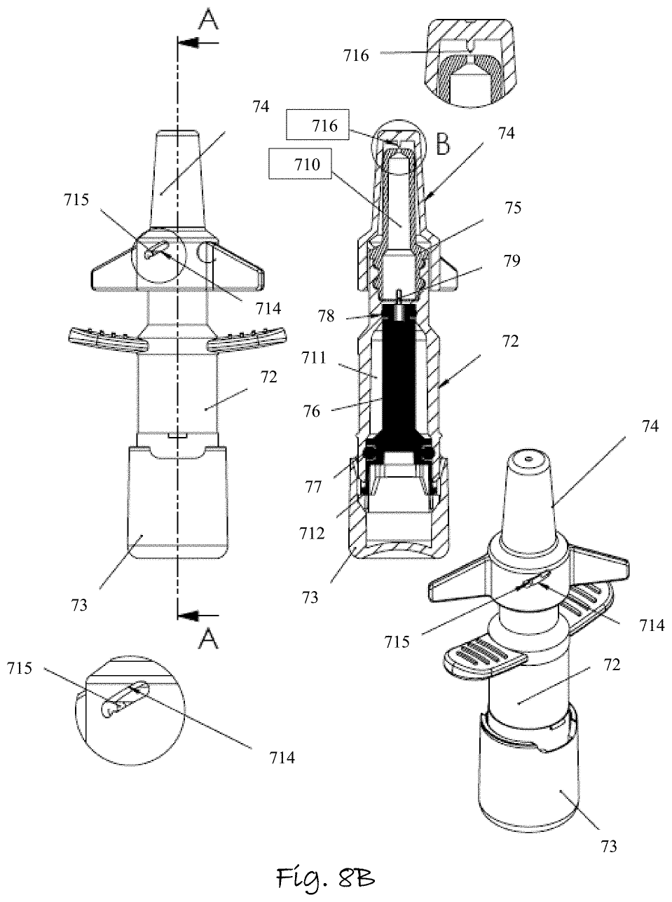

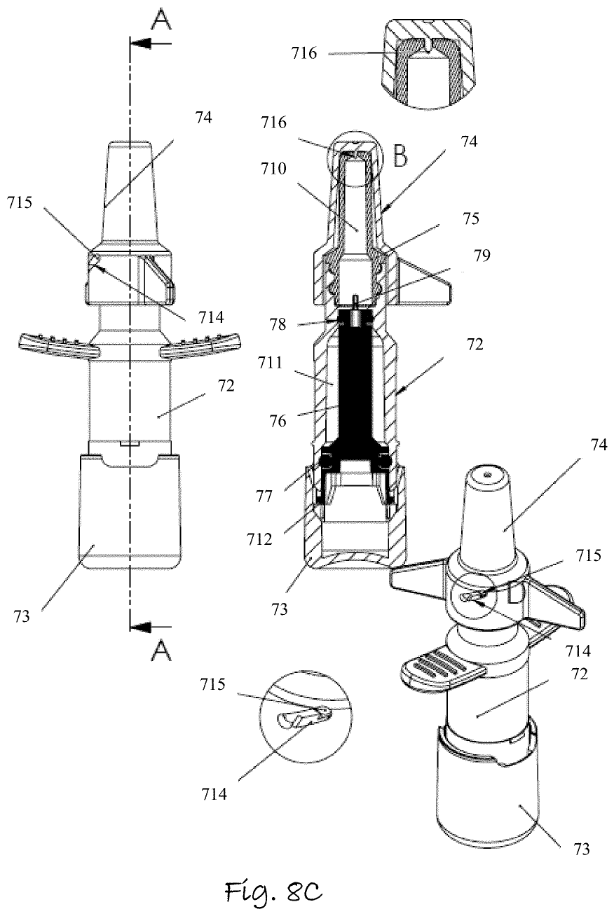

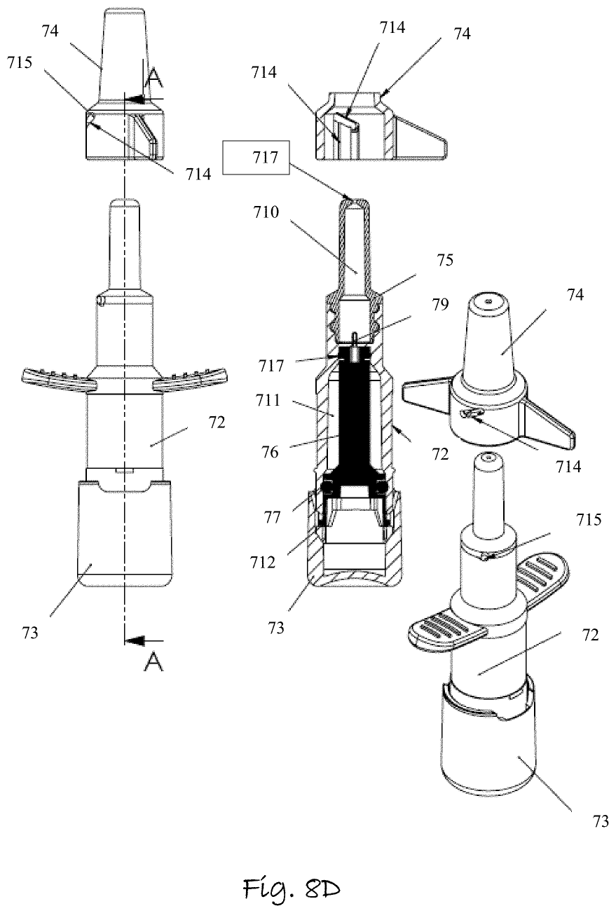

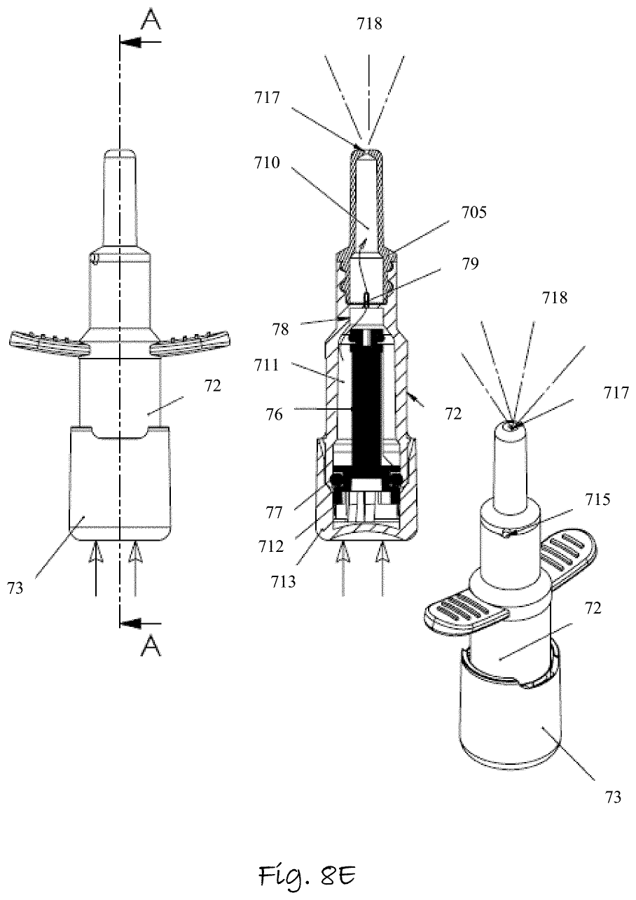

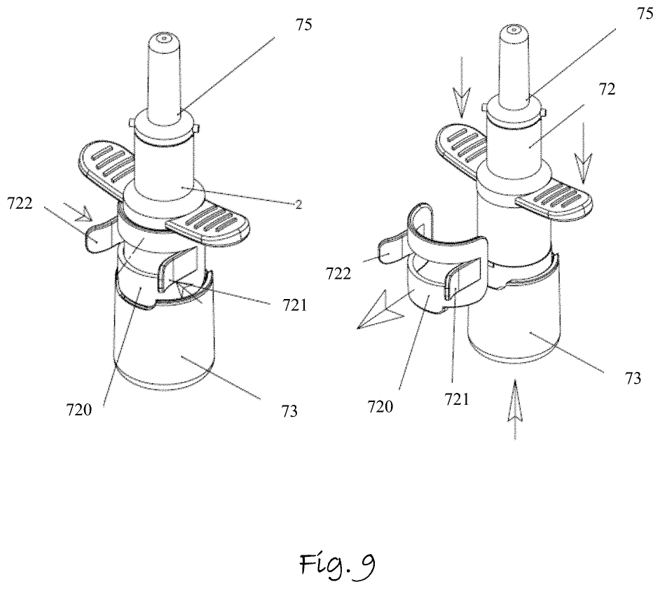

Reference is now made to FIG. 9 disclosing a device according to another embodiment of the present invention, where the device comprises a safety latch 720 with its two locks 721-722, configured to avoid undesired or accidental operation of the device, i.e., by pressing activation mechanism base 73 and pressurized fluid container (body) 72.

It is well in the scope of the invention wherein the pressurized fluid is accommodated within container for a respectively long time, e.g., by having a pre-pressurized container (step 1A) in a fluid connection (step 2A) with the BFS and releasing the same (step 3A), or alternatively a container suitable to pressuring the fluid in situ within the container, e.g., by introducing a pump or piston mechanism that pressuring ambient air to the container in a first step (step 1B) and accommodating the pressurized fluid along a relatively short time of step 2B, then free the fluid to flow in step 3B.

By the device disclosed herein, the pre-aerosolized mixture of gas and substance exits the device with a significant driving force as a mixture of aerosol and pre-aerosolized material (fluid or powder). When the pre-aerosolized material hits the walls of the nasal passages, it “explodes” into a fine aerosol that is capable of being driven by the pressure deep into the nasal passages to deposit in the desired region.

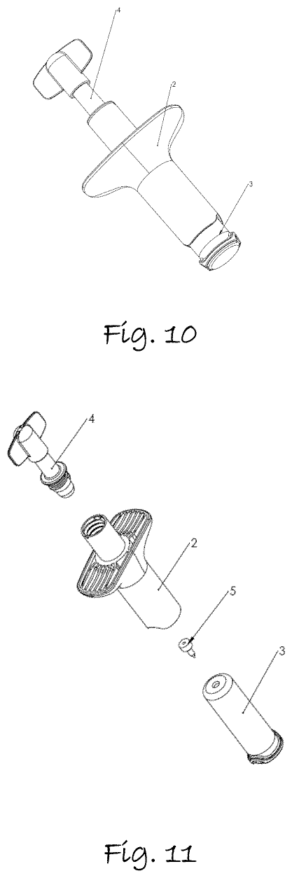

Reference is now made to FIGS. 10-11 which illustrates another embodiment of the present invention, in which the pierced container is the pressurized gas container.

FIG. 10 illustrates the nosepiece 4 (containing the medicament container), the handle, the device body 2 and the pressurized air container 3.

Reference is now made to FIG. 11, illustrating an embodiment in which the pressurized gas container 3 is pierced by means of a dedicated hollow needle 5.

According to this embodiment, once the device is activated (the pressurized gas container 3 is pressed against the handle), needle 5 pierces the pressurized gas container 3 and the pressurized gas exits therefrom and enters the medicament container to entrain the medicament into the nasal cavity.

Needle 5 is a hollow needle such that when the same pierces the gas container, the pressurized gas exit the pressurized fluid (e.g., gas) container 3 throughout needle 5.

Needle 5 comprises two ends, one of which is a flat, non-sharp end, in fluid communication with the medicament container (will be disclosed hereinafter) and the second one has a sharp end, adapted to pierce the gas container. The two ends interconnected by means of a hollow tube. Once the gas container is pierced the pressurized gas exits therefrom through the hollow tube.

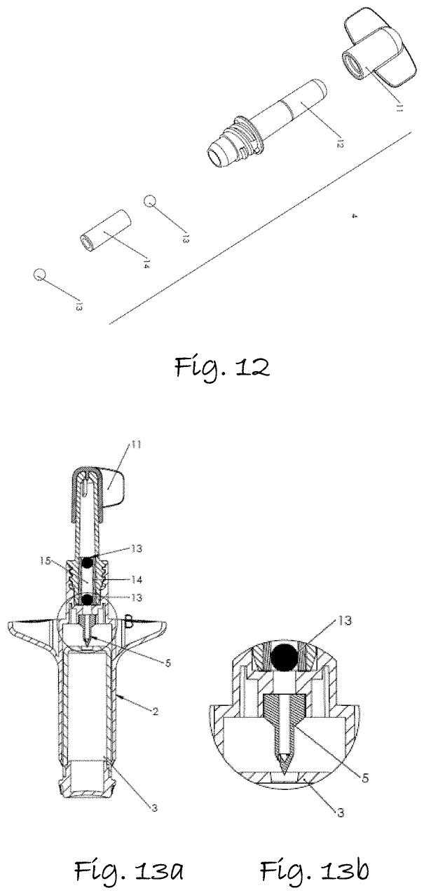

Reference is now made to FIG. 12 illustrating one embodiment of the nosepiece 4 enclosing a powder medicament. According to this embodiment the nosepiece has a cap 11 adapted to cover the distal most part of the nosepiece body 12. Within the nosepiece body 12, a dedicated medicament capsule 14 is disposed. The medicament capsule 12 is adapted to enclose e.g., powder medicament. However, one skilled in the art would acknowledge that also liquid, gas or gel medicament are also within the scope of the present invention. Furthermore, capsule 12 can enclose a combination of both liquid and powder medicaments.

According to this embodiment, at least one spherical element, preferably ball 13, is disposed within the medicament capsule 12. According to another embodiment, at least two spherical elements, preferably ball 13, are disposed within the medicament capsule 12, such that the medicament are disposed between the two balls 13.

The spherical elements are adapted to both (a) seal the medicament capsule 12 and prevent leakage of medicament therefrom; and, (b) once the pressurized air exits the pressurized gas container, the bottom most special element 13 is removed from its position (thereby removing the sealing thereof and enabling the pressurized gas to enter therein) and mixes/compress the medicament enclosed between the two spherical elements 13. Once, the first (bottom most) spherical element is removed, the same compress the medicament disposed between the first and second spherical elements 13 and cases the removal of the second (upper most) spherical element 13. Thereafter, the medicament along with the pressurized gas are delivered to the nasal cavity.

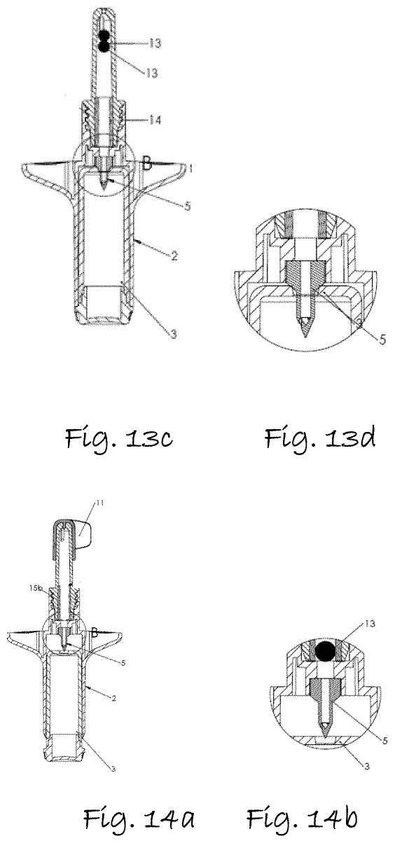

Reference is now made to FIGS. 13a-13d illustrating the step-by-step activation of the device. FIG. 13a illustrates the device before activation, in which the medicament is enclosed within the medicament container 14 and the gas container is not pierced yet. FIG. 13b provides a closer view of the needle 5 before activation thereof.

FIG. 13c illustrates the device after activation, in which the gas container is pierced, the pressurized gas had entered the medicament container, removed the two spherical elements 13 (the two spherical elements 13 are shown outside capsule 14) to enable delivery of the medicament 15. FIG. 13d provides a closer view of the needle 5 after activation thereof (i.e., pierced the gas container).

Reference is now made to FIGS. 14a-14b illustrating other embodiment of the present invention in which liquid medicament are utilized. According to this embodiment, only one spherical element 13 is utilized. Said spherical element 13 is disposed in the bottom most part of the medicament capsule to seal the same. Once, the needle 5 pierces the gas container, the pressurized gas exits the gas container through the hollow needle to remove the spherical element, entrain the medicament and deliver the same to the nasal cavity. FIG. 14a illustrates the device before activation, in which the medicament is enclosed within the medicament container 14 and the gas container is not pierced yet. FIG. 14b provides a closer view of the needle 5 before activation thereof.

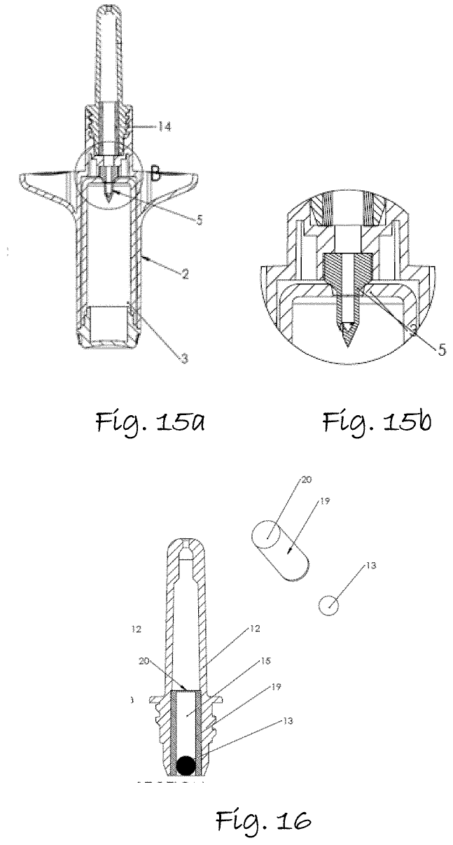

FIG. 15a illustrates the device after activation, in which the gas container is pierced, the pressurized gas had entered the medicament container, removed two spherical element 13 (the two spherical elements 13 are shown outside capsule 14) to enable delivery of the medicament 15. FIG. 15b provides a closer view of the needle 5 after activation thereof (i.e., pierced the gas container).

Reference is now made to FIG. 16, which illustrates a closer view of one embodiment of a capsule enclosing either liquid medicament or powder or 15 and the spherical element 13. According to one embodiment a combination of liquid medicament and powder can be enabled by displacing e.g., the powder medicament on the bottom part of the capsule and the liquid medicament in the upper part thereof and vice versa.

According to this embodiment, the capsule comprises the spherical element 13 being displaced in the bottom most part of the capsule (providing sealing in the bottom part) and a breakable membrane 20 in the upper part thereof (providing sealing in the upper part).

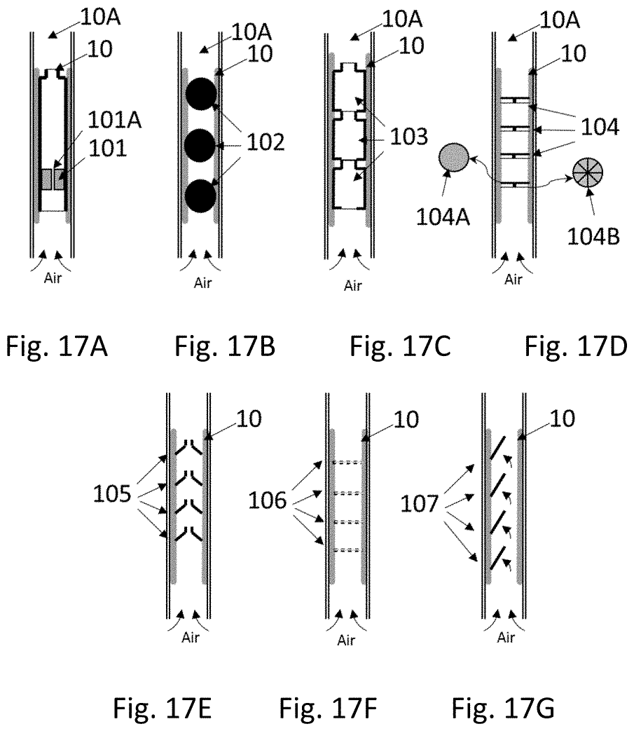

It should be noted that different embodiments of the capsule are available. Reference is now made to FIGS. 17a-17g.

FIG. 17A shows a plunger-type barrier (101) between compartments. In this exemplary embodiment, there is one plunger (101). In other embodiments, more or fewer plungers (101) can be present. The plunger (101) comprises a hole or slot small enough to prevent passage of substance therethrough, but wide enough to allow passage of compressed air therethrough. When the device is activated, compressed gas (curved arrows at bottom) enters the capsule (10). The pressure forces the plunger (101) upward, forcing substance above the plunger (101) out of the top of the capsule. Substance below the plunger (101) will be forced upward by the compressed air, to mix with the substance above the plunger in a nose piece (not shown). The plunger (101) passes through the top of the capsule into an intermediate space (10A) below the nosepiece (not shown; a shoulder or other barrier (not shown) prevents the plunger (101) from exiting the nosepiece.

The hole or slot (101A) in the plunger (101) is narrow enough to prevent substance leakage during storage, and wide enough to allow compressed gas passage during activation, wiping the substance from the container during activation. The hole or slot (101A) in the plunger (101) can be designed in many ways to allow delivery that is very efficient, having a residual volume of less than 15% of the original volume. The plunger (101) can be made either from a flexible materials such as, but not limited to, silicone, rubber, flexible plastic or from a hard material such as, but not limited to, a polymer such as Delrin®, a plastic, nylon, metal and any combination thereof.

FIG. 17B shows ball-type barriers (102) between compartments. The balls (102) provide both a separation function, before activation, and a mixing function during activation. In this exemplary embodiment, there are 3 balls (103). In other embodiments, more or fewer balls (103) can be present. When the device is activated, compressed gas (curved arrows at bottom) enters the capsule (10). The pressure forces the balls (102) upward, forcing substance above the topmost ball (102) out of the top of the capsule. The topmost ball (102) passes through the top of the capsule into an intermediate space (10A) below the nosepiece (not shown; a shoulder or other barrier (not shown) prevents the balls (102) from exiting the nosepiece. The substance between the first and second balls can then pass through the top of the capsule (10) into the nosepiece (not shown, and mix with the first substance. The second ball (102) can then enter the intermediate space (10A), and similarly with all balls (102) in the capsule (10) until the capsule (10) is empty.

Ball-type barriers (102) are useful when mixing of several components should occur only upon delivery, when one or more substance should be maintained at low humidity, when the viscosity of the substance varies significantly, and any combination thereof. In addition, contact between the ball (102) and the walls of the capsule (10) can also ensure effective release of the substance from the capsule (10). Examples of substances which tend to cling to walls include, but are not limited to, oils and some powders. The barriers can be balls, as in the embodiment shown, angular dividers or any other shape which can be easily moved by the released compressed gas (low-friction contacts), and still provide effective sealing between the elements to avoid mixing during, for example, shipment and storage.

FIG. 17C shows an embodiment with linked drug containers (103) within the capsule (10). In this exemplary embodiment, there are 3 linked drug containers (103). In other embodiments, more or fewer linked drug containers (103) can be present. The linked drug containers (103) are sealed by frangible membranes. A single frangible membrane can seal the top of one drug container (103) and the bottom of the adjacent drug container (103), separate frangible membranes (103) can be used for adjacent ends of drug containers, and any combination thereof. When the device is activated, compressed gas (curved arrows at bottom) enters the capsule (10). The pressure bursts the membranes, allowing mixing and exit into the nosepiece of the substance s within the linked drug containers (103).

In a preferred embodiment, each drug containers (103) is made of a soft thin sheet. The sheet can be a polymeric membrane, a continuous sheet or any other form which is thin enough to be easily torn when desired by the released of the compressed air. All drug containers (103) are connected to each other during manufacturing. Mixing occurs only during activation, with the compressed gas tearing the membranes/sheets dividing the compartments. Once the membranes are torn, the substance s are exposed to the compressed gas, mixed and delivered.

FIG. 17D shows an embodiment with sets of two-layer membranes (104A, 104B) within the capsule (10). In this exemplary embodiment, there are 4 sets of two-layer membranes (104A, 104B). In other embodiments, more or fewer sets of two-layer membranes (104A, 104B) can be present. The lower membrane (104B) is reticulated, with portions separable from each other, and the upper membrane (104A), frangible. When the device is activated, compressed gas (curved arrows at bottom) enters the capsule (10). The pressure causes the separable portions of the lower membrane (104B) to rotate upward, tearing the upper membrane (104A) and allowing mixing and exit into the nosepiece of the substance s within the capsule (10).

This embodiment differs from the previous one in that: (a) the drug containers do not form one unit; (b) the separate zones are separated from each other by membrane which is composed of two layers: one provides the rigidity of the membrane and is made of a rigid material, and the other one is a continuous flexible sheet which seals against the lower rigid part during until activation and which opens when air is pressed against its lower side The membranes (104A, 104B) open only one way, when air presses against their lower side during activation, allowing mixing of the substances during delivery.

FIG. 17E shows an embodiment with duckbill valves (105) within the capsule (10). In this exemplary embodiment, there are 4 duckbill valves (105). In other embodiments, more or fewer duckbill valves (105) can be present. When the device is activated, compressed gas (curved arrows at bottom) enters the capsule (10). The pressure causes the duckbill valves (105) to rotate upward, allowing exit and mixing of the substance s within the capsule (10).

FIG. 17F shows an embodiment with frangible membranes (105) within the capsule (10). In this exemplary embodiment, there are 4 frangible membranes (105). In other embodiments, more or fewer frangible membranes (105) can be present. When the device is activated, compressed gas (curved arrows at bottom) enters the capsule (10). The pressure causes the frangible membranes (105) to tear, allowing mixing and exit into the nosepiece (not shown) of the substance s within the capsule (10).

FIG. 17G shows an embodiment with bendable membranes (106) within the capsule (10). In this exemplary embodiment, there are 4 bendable membranes (106). In other embodiments, more or fewer bendable membranes (106) can be present. When the device is activated, compressed gas (curved arrows at bottom) enters the capsule (10). The pressure causes the bendable membranes (106) to rotate upward (curved arrows in middle) about connection points between the bendable membranes (106) and the capsule (10) wall, allowing mixing and exit into the nosepiece (not shown) of the substances within the capsule (10).

These exemplary embodiments allow holding the substances separate during storage and mixing the substances only upon activation and delivery. In some embodiments, the device or the substances therein can be configured to generate a temperature change, either heating or cooling, during mixing and delivery. The device can further be configured so that components for creating a temperature change in the device are not released with the delivered substances.

Heating and cooling can be triggered by mechanical force, by pressure, by chemical reaction and any combination thereof. This can be done inside the drug capsule, around the drug capsule, or outside the device itself in its packaging, to be triggered right before activation of the device.

Such temperature change can be generated during activation (short time temperature change) or prior to activation (long time temperature change). Long time temperature changes require a temperature activation separated from the delivery activation.

Either option, or at least the long time temperature change, further requires proper device sealing to allow temperature to be maintained inside the device and to allow equilibration prior delivery. Such options can further include a temperature indicator, such as by a color change in a dedicated control window, to allow the user to know that the device is ready for activation.

A temperature change can be an increase in temperature, a decrease of temperature, or both.

A temperature change can be useful for example for:
Substance mixing
Dissolution of one substance in another
Absorption of a substance or mixture of substances in tissue, for example, a delivery temperature regulated with respect to the temperature of the nasal passages.
Effective scattering of a substance or mixture of substances on tissue, for example, to create a flat, thin, uniform layer in the nasal passages and hence improve absorption
Affect the viscosity of a substance or mixture of substances (both increase and decrease of viscosity can occur).
Affect nature of a substance or mixture of substances. For example: polymerization can be initiated only during delivery, or during or after contact with tissue.

One embodiment comprises two heating agents. These heating agents are in compartments of a capsule. Upon activation of the device, or upon activation of heating (for example, buy pressing a button), a membrane separating the two compartments is torn, allowing the heating agents to mix and to generate heat within the device. Other membranes are not torn by this activity, which keeps the heating agents in a sealed compartment—sealed so as to prevent delivery of heating agent delivery but allow gas passage to other compartments. Passage of the compressed gas then delivers the heated substances or other desired substances. Mixing, as disclosed above, can occur during delivery.

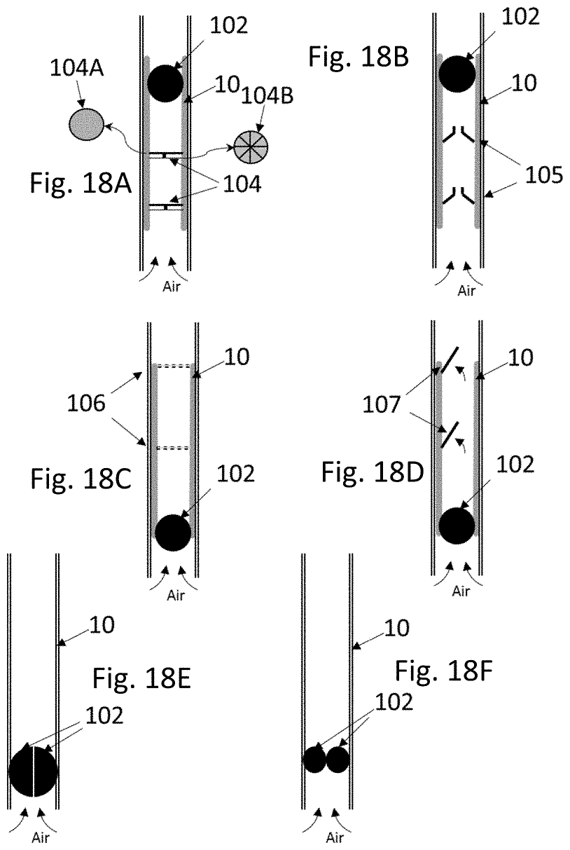

Reference is now made to FIGS. 18a-18f illustrating another embodiment of the capsule.

FIG. 18A shows an embodiment with sets of two-layer membranes (104A, 104B) and a mixing ball (102) within the capsule (10). In this exemplary embodiment, there are 2 sets of two-layer membranes (104A, 104B) and a single mixing ball (102) at the top of the capsule (10). In other embodiments, more or fewer sets of two-layer membranes (104A, 104B) and more or fewer mixing balls (102) can be present; the mixing balls (102) can be at any desired location within the capsule (10). The lower membrane (104B) is reticulated, with portions separable from each other, and the upper membrane (104A), frangible. When the device is activated, compressed gas (curved arrows at bottom) enters the capsule (10). The pressure causes the separable portions of the lower membrane (104B) to rotate upward, tearing the upper membrane (104A) and allowing mixing and exit into the nosepiece of the substances within the capsule (10). Further mixing is provided by the mixing ball (102). As disclosed above, a shoulder or other stopper in the nosepiece (not shown) prevents the mixing ball (102) from exiting the nosepiece (not shown).

FIG. 18B shows an embodiment with duckbill valves (105) and a mixing ball (102) within the capsule (10). In this exemplary embodiment, there are 2 duckbill valves (105) and a single mixing ball (102) at the top of the capsule (10). In other embodiments, more or fewer duckbill valves (105) can be present and more or fewer mixing balls (102) can be present; the mixing balls (102) can be at any desired location within the capsule (10). When the device is activated, compressed gas (curved arrows at bottom) enters the capsule (10). The pressure causes the duckbill valves (105) to rotate upward, allowing exit and mixing of the substances within the capsule (10). Further mixing is provided by the mixing ball (102). As disclosed above, a shoulder or other stopper in the nosepiece (not shown) prevents the mixing ball (102) from exiting the nosepiece (not shown).

FIG. 18C shows an embodiment with frangible membranes (105) and a mixing ball (102) within the capsule (10). In this exemplary embodiment, there are 4 frangible membranes (105) and a single mixing ball (102) at the top of the capsule (10). In other embodiments, more or fewer frangible membranes (105) can be present and more or fewer mixing balls (102) can be present; the mixing balls (102) can be at any desired location within the capsule (10). When the device is activated, compressed gas (curved arrows at bottom) enters the capsule (10). The pressure causes the frangible membranes (105) to tear, allowing mixing and exit into the nosepiece (not shown) of the substances within the capsule (10). Further mixing is provided by the mixing ball (102). As disclosed above, a shoulder or other stopper in the nosepiece (not shown) prevents the mixing ball (102) from exiting the nosepiece (not shown).

FIG. 18D shows an embodiment with bendable membranes (106) and a mixing ball (102) within the capsule (10). In this exemplary embodiment, there are 4 bendable membranes (106) and a single mixing ball (102) at the top of the capsule (10). In other embodiments, more or fewer bendable membranes (106) can be present and more or fewer mixing balls (102) can be present; the mixing balls (102) can be at any desired location within the capsule (10). When the device is activated, compressed gas (curved arrows at bottom) enters the capsule (10). The pressure causes the bendable membranes (106) to rotate upward (curved arrows in middle) about connection points between the bendable membranes (106) and the capsule (10) wall, allowing mixing and exit into the nosepiece (not shown) of the substances within the capsule (10). Further mixing is provided by the mixing ball (102). As disclosed above, a shoulder or other stopper in the nosepiece (not shown) prevents the mixing ball (102) from exiting the nosepiece (not shown).

FIG. 18E shows an embodiment with two half balls (102). In this exemplary embodiment, there is one pair of half-balls (102). In other embodiments, more pairs of half-balls (102) can be present. When the device is activated, compressed gas (curved arrows at bottom) enters the capsule (10). The pressure causes the half-balls (102) to move upward. They will separate and tumble as they move, allowing gas to pass between and around them thus mixing and delivering the substance. As disclosed above, a shoulder or other stopper in the nosepiece (not shown) prevents the mixing ball (102) from exiting the nosepiece (not shown).

FIG. 18F shows an embodiment with two attached mixing balls (102). In other embodiments, more mixing balls (102) can be present. When the device is activated, compressed gas (curved arrows at bottom) enters the capsule (10). The pressure causes the mixing balls (102) to move upward, thus causing efficient mixing of the substances. As disclosed above, a shoulder or other stopper in the nosepiece (not shown) prevents the mixing ball (102) from exiting the nosepiece (not shown).

The mixing balls need not be spherical; any shape that will provide good sealing during storage and low-friction movement during activation can be used.

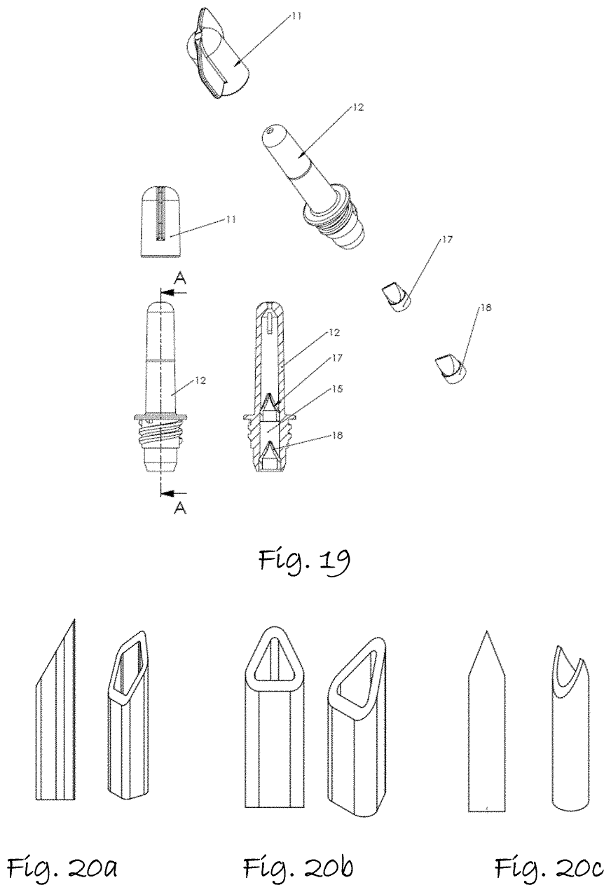

Reference is now made to FIG. 19, which illustrates a closer view of another embodiment of the capsule. According to this embodiment, the capsule comprises at least one uni-directional valve such as duckbill valve. In the embodiment illustrated in FIG. 19 two uni-directional valve (e.g., duckbill valves), 17 and 18 are provided, where the medicament 15 is disposed therebetween.

As noted above, the medicament delivered can either powder, liquids, gas, gel and any mixing thereof.

It should be noted that the medicament delivered can either powder, liquids, gas, gel and any mixing thereof.

Reference is now made to FIGS. 20a-20d illustrating different shapes of hollow needle. As can be seen from the figs. the sharp end of the needle can have a cross section of any shape (e.g., rectangular, triangular, pentagon etc.). Each wig of the needle can accommodate at least one orifice, throughout which the pressurized gas can exit and enter the capsule and entrain the medicament to be delivered to the nasal cavity.

According to any of the embodiment as described above, the pressurized gas container can be mad of at least one material selected from a group consisting of high- or low-density polyethylene, high- or low-density polypropylene, any plastic resin, glass, plastics, Aluminum and any combination thereof.

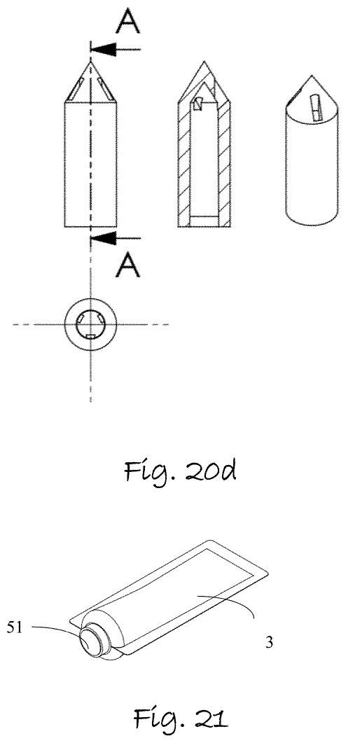

Reference is now made to FIG. 21 illustrating another embodiment of the pressurized fluid (e.g., gas) container 3.

According to another embodiment, the pressurized fluid (e.g., gas) container (enclosing the pressurized and compressed fluid) is made of material being a high barrier film. According to one embodiment the high barrier film is a high barrier Aluminum film.

According to this embodiment, the high barrier film (e.g., Aluminum film) is ultrasonic soldered to create the pressurized-fluid container. Such ultrasonic soldering of the Aluminum high barrier film results in an airtight container that prevents any air leakage of the compressed and pressurized gas therewithin.

Such container 3 could be sealed by a breakable membrane 51 (that breaks once the pressure of the pressurized gas, enclosed therewithin, reaches a predetermined level), or septum 51, or any other sealable means.

In the foregoing description, embodiments of the invention, including preferred embodiments, have been presented for the purpose of illustration and description. They are not intended to be exhaustive or to limit the invention to the precise form disclosed. Obvious modifications or variations are possible in light of the above teachings. The embodiments were chosen and described to provide the best illustration of the principals of the invention and its practical application, and to enable one of ordinary skill in the art to utilize the invention in various embodiments and with various modifications as are suited to the particular use contemplated. All such modifications and variations are within the scope of the invention as determined by the appended claims when interpreted in accordance with the breadth they are fairly, legally, and equitably entitled.

1. A device for delivering either one or more substances within at least one body cavity, comprising:
(i) at least one nosepiece comprising at least one capsule comprising V_{sub} [ml or mg] of said substances; said capsule having at least one fluid inlet port of diameter Dᵢₙ [mm] and at least one fluid discharging outlet of diameter Dₒᵤₜ [mm], configured for placement in proximity to said body cavity;
(ii) at least one base in communication with said at least one nosepiece, said at least one base comprises at least one chamber configured to confine compress and pressurized fluid at volume V_{PF} [ml] and pressure P_{PF} [barg];
(iii) at least one hollow puncturing member; said at least one hollow puncturing member is characterized by at least one end in fluid communication with said at least one capsule in said nosepiece and a second end in fluid communication with said at least one base; said first and second ends are fluidly interconnected by a hollow tube; said fluid inlet port of said capsule is configured by means of size and shape to interface in a sealable manner with said one end of said at least one puncturing member;
wherein actuation of said base configured to enable piercing of said chamber, by means of said puncturing member to enables the pressurized fluid to exit said chamber and entrain through said hollow tube of said hollow puncturing member to said capsule, entrain said substance and deliver the same to said body cavity.

2. The device of claim 1, wherein said chamber is made of material being a high barrier film.

3. The device of claim 2, wherein said high barrier film is a high barrier Aluminum film.

4. The device of claim 1, wherein actuation of said base is pressing on the same.

5. The device of claim 1, wherein said nosepiece comprising at least one nosepiece cover configured by means of size and shape to cover, in a sealable manner, at least partially said nosepiece

6. The device of claim 1, wherein said nosepiece cover and said nosepiece are coupled to each other.

7. The device of claim 6, wherein said coupling between nosepiece cover and said nosepiece said is reversible.

8. The device of claim 1, wherein said nosepiece comprises at least one port throughout which said at least one substance exits said device, such that said nosepiece cover seals said at least one port and removal thereof removes said seal.

9. The device of claim 1, wherein said capsule is selected from a group consisting of pierceable container, a blow-fill-seal and a form-fill-seal and any combination thereof.

10. The device of claim 1, wherein said at least one chamber is a container adapted to hold said pressured fluid at said P_{PF} for prolong periods of time.

11. The device of claim 1, wherein capsule is made of at least one material selected from a group consisting of high- or low-density polyethylene, high- or low-density polypropylene, any plastic resin, glass and any combination thereof.

12. The device of claim 1, wherein said one end of said at least one hollow puncturing member and said capsule are formed as a single unit, such that the lower surface of said capsule is integrated with said one end of said at least one hollow puncturing member.

13. The device of claim 1, wherein said volume V_{PF} [ml] of said pressurized fluid at pressure P_{PF} [barg] is released from said chamber within a short period of time, <500 milliseconds (dT), via said fluid inlet port, entrains said substances, erupts via said fluid discharging outlet port into said body cavity, such that the release time of said V_{sub} [ml or mg] of said substances and said V_{PF} [ml] of said pressurized fluid, dTᵣₑₗₑₐₛₑ is less than 500 milliseconds.

14. The device of claim 1, wherein said device is configured to deliver said V_{sub} substance and V_{PF} pressurized fluid through said fluid discharging outlet of diameter D [mm] wherein at least one of the following is held true:
a. V_{PF} is in a range of 1 to 50 ml;
b. V_{sub} is in a range of about 0.01 to about 7 ml;
c. Dᵢₙ and/or Dₒᵤₜ is in a range of 0.2 to 6 mm;
d. P_{PF} is in a range of about 0 to about 10 barg;
e. said pressure rate,
$$\frac{dP}{dT} \to ∞ ;$$
f. said pressure velocity is greater than 0.001 barg/ms;
g. said pressure velocity is greater than 0.01 barg/ms;
h. said volume rate dV_{sub}/dT or dV_{sub}/dTᵣₑₗₑₐₛₑ is greater than 0.0001 ml/ms;
i. said volume rate dV_{sub}/dT or dV_{sub}/dTᵣₑₗₑₐₛₑ is greater than 0.001 ml/ms;
j. said volume rate dV_{PF}/dT or dV_{PF}/dTᵣₑₗₑₐₛₑ is greater than 0.001 ml/ms;
k. said volume rate dV_{PF}/dT or dV_{PF}/dTᵣₑₗₑₐₛₑ is greater than 0.01 ml/ms;
l. any combination thereof.

15. The device of claim 1, wherein at least one of the following is true:
a. said body cavity is selected from a group consisting of nasal cavity, the mouth, the throat, an ear, the eye, the vagina, the rectum, the urethra, and any combination thereof.
b. said pressurized gas is selected from a group consisting of air, nitrogen, oxygen, carbon dioxide, helium, neon, xenon, nitric oxide and any combination thereof;
c. during dispensing of said at least one substance, a mixture of said predetermined volume V_{gas} [ml] of said pressurized gas with said predetermined volume V_{sub} [ml or mg] of said substance entrained within it forms a plume of aerosol; said aerosol having a predetermined distribution, said distribution being either homogeneous or heterogeneous, said heterogeneous distribution is selected from a group consisting of: an arbitrary distribution, a distribution in which the density of said at least one substance within said mixture follows a predetermined pattern, and any combination thereof; characteristics of said aerosol selected from a group consisting of: particle size, particle shape, particle distribution, and any combination thereof, are determinable from characteristics of said device selected from a group consisting of: said predetermined volume of said pressurized gas, said predetermined volume of said substance, said predetermined pressure of said pressurized gas, said predetermined orifice size, and any combination thereof;
d. at least one said substance is selected from a group consisting of a gas, a liquid, a powder, an aerosol, a slurry, a gel, a suspension and any combination thereof;
e. at least one said substance is stored under either an inert atmosphere or under vacuum to prevent reactions during storage;
f. a dose-response curve is substantially linear for brain concentration of said substance when administered nasally via said device; and
g. a dose-response curve for brain concentration having a fit selected from a group consisting of logarithmic, parabolic, exponential, sigmoid, power-low, and any combination thereof; of said substance when administered nasally via said device.

16. The device of claim 1, wherein said nosepiece cover configured to provide an air-tight closure for said port, said port cover slideable along said device, rotatable around said device, rotatable around a hinge on the exterior of said device and any combination thereof.

17. The device of claim 1, further comprising a safety latch, adapted to prevent accidental operation of said device.

18. The device of claim 1, wherein said substance is selected from a group consisting of proteins; stem-cells; cells, organs, portions, extracts, and isolations thereof; macro-molecules; RNA or other genes and proteins-encoding materials; neurotransmitters; receptor antagonists; hormones; Ketamine; Baqsimi product commercially available by Lilly (US); Glucagon; substrates to treat one of eth followings: anaphylaxis, Parkinson, seizures and opioid overdose; epinephrine; atropine; metoclopramide; commercially available Naloxone or Narcan products; Esketamine (Spravato); Radicava [edaravone]; Ingrezza [valbenazine]; Austedo [deutetrabenazine]; Ocrevus [ocrelizumab]; Xadago [safinamide]; Spinraza [nusinersen]; Zinbryta [daclizumab]; Nuplazid [pimavanserin]; Aristada [aripiprazole lauroxil]; Vraylar [cariprazine]; Rexulti [brexpiprazole]; Aptiom [eslicarbazepine acetate]; Vizamyl [flutemetamol F18 injection]; Brintellix [vortioxetine]; Tecfidera [dimethyl fumarate]; Dotarem [gadoterate meglumine]; Antibody mediated brain targeting drug delivery including aducanumab, gantenerumab, bapineuzumab, solanezumab, ofatumumab CD20, BIIB033, LCN2, HMGB1; insulin; oxytocin; orexin-A; leptin; benzodiazepine, midazolam; naloxone; perillyl alcohol; camptothecin; phytochemicals including curcumin and chrysin; nucleotides; olanzapine; risperidone; Venlafaxin; GDF-5; zonisamide; ropinirole; plant-originated and synthetically-produced terpenes and cannabinoids, including THC and CBD; valproric acid; rivastigmine; estradiol; topiramate or an equivalent preparation comprising CAS No. 97240-79-4; MFSD2 or MFSD2A or sodium-dependent lysophosphatidylcholine symporter, midazolam; naloxone; perillyl alcohol; camptothecin; phytochemicals including curcumin and chrysin; nucleotides; olanzapine; risperidone; Venlafaxin; GDF-5; zonisamide; ropinirole; plant-originated and synthetically-produced terpenes and cannabinoids, including THC and CBD; valproric acid; rivastigmine; estradiol; topiramate or an equivalent preparation comprising CAS No. 97240-79-4; MFSD2 or MFSD2A or sodium-dependent lysophosphatidylcholine symporter; and any esters, salts, derivatives, mixtures, combinations thereof, with or without a carrier, liposomes, lyophilic or water-miscible solvents, surfactants, cells, cells fractions, at a therapeutically effective concentration.

19. The device of claim 1, wherein said capsule is a hollow tube characterized by at least two ends interconnect to each other, at least one of which is positioned proximal to said chamber.

20. The device of claim 19, wherein said capsule comprises at least one spherical element positioned by at least one of said ends, adapted to seal said capsule and prevent leakage of said at least one substrate therefrom.

21. The device of claim 20, wherein said capsule comprises two spherical elements, each of which is disposed at each of said ends, such that said at least one substrate is positioned therebetween.

22. The device of claim 21, wherein said two spherical elements, once said pressurized fluid exits said chamber, are adapted to mix said at least one substrate and said at least one substrate.

23. The device of claim 19, wherein said capsule comprises at least one membrane positioned by at least one of said ends, adapted to seal said capsule and prevent leakage of said at least one substrate therefrom.

24. The device of claim 23, wherein said capsule comprises two membranes, each of which is disposed at each of said ends, such that said at least one substrate is positioned therebetween.

25. The device of claim 19, wherein said capsule comprises at least one duckbill valve positioned by at least one of said ends, adapted to seal said capsule and prevent leakage of said at least one substrate therefrom.

26. The device of claim 25, wherein said capsule comprises two duckbill valves, each of which is disposed at each of said ends, such that said at least one substrate is positioned therebetween.

27. The device of claim 19, wherein said capsule comprises at least one spherical element, membrane, uni-directional valve, duckbill valve and any combination thereof.

28. A method for delivering either one or more substances within at least one body cavity, characterized by steps of
a. providing:
i. at least one nosepiece comprising at least one capsule comprising V_{sub} [ml or mg] of said substances; said capsule having at least one fluid inlet port of diameter Dᵢₙ [mm] and at least one fluid discharging outlet of diameter Dₒᵤₜ [mm], configured for placement in proximity to said body cavity;
ii. at least one base in communication with said at least one nosepiece, said at least one base comprises at least one chamber configured to confine compressed and pressurized fluid at volume V_{PF} [ml] and pressure P_{PF} [barg];
iii. at least one hollow puncturing member; said at least one hollow puncturing member is characterized by at least one end in fluid communication with said at least one nosepiece and a second end in fluid communication with said at least one base; said first and second ends are fluidly interconnected by a hollow tube; said fluid inlet port of said capsule is configured by means of size and shape to interface in a sealable manner with said one end of said at least one puncturing member;
b. actuating said base thereby piercing of said chamber, by said at least one puncturing member.

29. The method of claim 28, wherein said chamber is made of material being a high barrier film.

30. The method of claim 29, wherein said high barrier film is a high barrier Aluminum film.

31. The method of claim 28, wherein step (b) of actuating said base additionally comprising pressing said base.

32. The method of claim 28, wherein step (b) of actuating said base enables the pressurized fluid to exit said chamber and entrain through said hollow tube of said hollow puncturing member to said capsule, entrain said substance and deliver the same to said body cavity.

33. The method of claim 28, wherein step (b) of actuating said base results in releasing said volume V_{PF} [ml] of said pressurized fluid at pressure P_{PF} [barg] within a short period of time, <500 milliseconds (dT); out of said chamber, via said fluid inlet thereby entraining said substances and erupting via said fluid discharging outlet into said body cavity, such that the release time of said V_{sub} [ml or mg] of said substances and said V_{PF} [ml] of said pressurized fluid, dTᵣₑₗₑₐₛₑ is less than 500 milliseconds.

34. The method of claim 28, wherein said nosepiece comprising a nosepiece cover and said nosepiece are coupled to each other.

35. The method of claim 28, wherein said coupling between nosepiece cover and said nosepiece said is reversible.

36. The method of claim 28, wherein removal of said nosepiece cover results in piercing said capsule to provide said fluid discharging outlet.

37. The method of claim 28, wherein removal of said nosepiece cover is obtained by at least one action selected from a group consisting of sliding said nosepiece cover along said device, rotating said nosepiece cover around said device, rotating said nosepiece cover around a hinge on the exterior of said device and any combination thereof.

38. The method of claim 28, wherein said nosepiece cover comprises at least one nosepiece puncturing member adapted to pierce said capsule to enable said fluid discharging outlet.

39. The method of claim 28, wherein said nosepiece comprises at least one port throughout which said at least one substance exits said device, such that said nosepiece cover seals said at least one port and removal thereof removes said seal.

40. The method of claim 28, wherein said capsule is selected from a group consisting of pierceable container, a blow-fill-seal and a form-fill-seal and any combination thereof.

41. The method of claim 28, wherein said at least one chamber is a container adapted to hold said pressured fluid at said P_{PF} for prolong periods of time.

42. The method of claim 41, wherein capsule is made of at least one material selected from a group consisting of high- or low-density polyethylene, high- or low-density polypropylene, any plastic resin, glass and any combination thereof.

43. The method of claim 28, wherein said device is configured to deliver said V_{sub} substance and V_{PF} pressurized fluid through said fluid discharging outlet of diameter D [mm] wherein at least one of the following is held true:
a. V_{PF} is in a range of 1 to 50 ml;
b. V_{sub} is in a range of about 0.01 to about 7 ml;
c. Dᵢₙ and/or Dₒᵤₜ is in a range of 0.2 to 6 mm;
d. P_{PF} is in a range of about 0 to about 10 barg;
e. said pressure rate,
$$\frac{dP}{dT} \to ∞ ;$$
f. said pressure velocity is greater than 0.001 barg/ms;
g. said pressure velocity is greater than 0.01 barg/ms;
h. said volume rate dV_{sub}/dT or dV_{sub}/dTᵣₑₗₑₐₛₑ is greater than 0.0001 ml/ms;
i. said volume rate dV_{sub}/dT or dV_{sub}/dTᵣₑₗₑₐₛₑ is greater than 0.001 ml/ms;
j. said volume rate dV_{PF}/dT or dV_{PF}/dTᵣₑₗₑₐₛₑ is greater than 0.001 ml/ms;
k. said volume rate dV_{PF}/dT or dV_{PF}/dTᵣₑₗₑₐₛₑ is greater than 0.01 ml/ms; and
l. any combination thereof.

44. The method of claim 28, additionally comprising at least one of the following steps:
a. selecting said body cavity from a group consisting of a nasal cavity, the mouth, the throat, an ear, the eye, the vagina, the rectum, the urethra, and any combination thereof;
b. selecting said gas from a group consisting of: air, nitrogen, oxygen, carbon dioxide, helium, neon, xenon, nitric oxide and any combination thereof;
c. dispensing said at least one substance, and during said step of dispensing, forming a plume of aerosol with predetermined distribution from a mixture of said predetermined volume V_{gas} [ml] of said pressurized gas and said predetermined volume V_{sub} [ml or mg] entrained within it; selecting said predetermined distribution from a group consisting of: a homogeneous distribution, a heterogeneous distribution; selecting said heterogeneous distribution from a group consisting of: an arbitrary distribution, a distribution in which the density of said at least one substance within said mixture follows a predetermined pattern, and any combination thereof; selecting characteristics of said aerosol from a group consisting of: particle size, particle shape, particle distribution, and any combination thereof, are determinable from characteristics of said device selected from a group consisting of: said predetermined volume of said pressurized gas, said predetermined volume of said substance, said predetermined pressure of said pressurized gas, said predetermined orifice size, and any combination thereof;
d. selecting said substance from a group consisting of: a gas, a liquid, a powder, a slurry, a gel, a suspension, and any combination thereof;
e. storing at least one said substance under either an inert atmosphere or under vacuum, thereby preventing reactions during storage; and
f. characterizing a dose-response curve for brain concentration of said substance to be of substantially linear form; and
g. a dose-response curve for brain concentration having a fit selected from a group consisting of logarithmic, parabolic, exponential, sigmoid, power-low, and any combination thereof; of said substance when administered nasally via said device.

45. The method of claim 28, wherein said substance is selected from a group consisting of proteins; stem-cells; cells, organs, portions, extracts, and isolations thereof; macro-molecules; RNA or other genes and proteins-encoding materials; neurotransmitters; receptor antagonists; hormones; Ketamine; Baqsimi product commercially available by Lilly (US); Glucagon; substrates to treat one of eth followings: anaphylaxis, Parkinson, seizures and opioid overdose; epinephrine; atropine; metoclopramide; commercially available Naloxone or Narcan products; Esketamine (Spravato); Radicava [edaravone]; Ingrezza [valbenazine]; Austedo [deutetrabenazine]; Ocrevus [ocrelizumab]; Xadago [safinamide]; Spinraza [nusinersen]; Zinbryta [daclizumab]; Nuplazid [pimavanserin]; Aristada [aripiprazole lauroxil]; Vraylar [cariprazine]; Rexulti [brexpiprazole]; Aptiom [eslicarbazepine acetate]; Vizamyl [flutemetamol F18 injection]; Brintellix [vortioxetine]; Tecfidera [dimethyl fumarate]; Dotarem [gadoterate meglumine]; Antibody mediated brain targeting drug delivery including aducanumab, gantenerumab, bapineuzumab, solanezumab, ofatumumab CD20, BIIB033, LCN2, HMGB1; insulin; oxytocin; orexin-A; leptin; benzodiazepine i.e. midazolam; naloxone; perillyl alcohol; camptothecin; phytochemicals including curcumin and chrysin; nucleotides; olanzapine; risperidone; Venlafaxin; GDF-5; zonisamide; ropinirole; plant-originated and synthetically-produced terpenes and cannabinoids, including THC and CBD; valproric acid; rivastigmine; estradiol; topiramate or an equivalent preparation comprising CAS No. 97240-79-4; MFSD2 or MFSD2A or sodium-dependent lysophosphatidylcholine symporter; and any esters, salts, derivatives, mixtures, combinations thereof, with or without a carrier, liposomes, lyophilic or water-miscible solvents, surfactants, cells, cells fractions, at a therapeutically effective concentration.

46. The method of claim 28, wherein said puncturing member comprising a plurality of holes through which said pressurized fluid exits said chamber and entrains said substance, after activation of said activation mechanism.

47. The method of claim 28, wherein said capsule is a hollow tube characterized by at least two ends interconnected to each other, at least one of which is positioned proximal to said chamber.

48. The method of claim 47, wherein said capsule comprises at least one spherical element position at one or more of said ends, adapted to seal said capsule and prevent leakage of said at least one substrate therefrom.

49. The method of claim 47, wherein said capsule comprises two spherical elements, each of which is disposed at each of said ends, such that said at least one substrate is positioned therebetween.

50. The method of claim 47, wherein said two spherical elements, once said pressurized fluid exits said chamber, are adapted to mix said at least one substrate and said at least one substrate.

51. The method of claim 28, wherein said capsule comprises at least one membrane positioned at one or more of said ends, adapted to seal said capsule and prevent leakage of said at least one substrate therefrom.

52. The method of claim 51, wherein said capsule comprises two membranes, each of which is disposed at each of said ends, such that said at least one substrate is positioned therebetween.

53. The method of claim 47, wherein said capsule comprises at least one duckbill valve positioned at one or more of said ends, adapted to seal said capsule and prevent leakage of said at least one substrate therefrom.

54. The method of claim 53, wherein said capsule comprises two duckbill valves, each of which is disposed at each of said ends, such that said at least one substrate is positioned therebetween.

55. The method of claim 47, wherein said capsule comprises at least one spherical element, membrane, duckbill valve and any combination thereof.

56. The device of claim 1, wherein said at least one hollow puncturing member comprises at least one orifice through which said pressurized fluid enters said hollow puncturing member.

57. The device of claim 56, wherein, once said device is actuated, said pressurized fluid exits said chamber and enters said hollow puncturing member through said at least one orifice.

58. The method of claim 28, wherein said at least one hollow puncturing member comprises at least one orifice through which said pressurized fluid enters said hollow puncturing member.

59. The method of claim 58, wherein, once said device is actuated, said pressurized fluid exits said chamber and enters said hollow puncturing member through said at least one orifice.
